(19) United States
(12) Patent Application Publication
(10) Pub. No.: US 2009/0105588 A1
(43) Pub. Date: Apr. 23, 2009

(54) Real-Time Ultrasound Monitoring of Heat-Induced Tissue Interactions

(75) Inventors: Stanislav Emelianov, Austin, TX (US); Thomas Milner, Austin, TX (US); Jignesh Shah, Austin, TX (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 12/238,753

(22) Filed: Sep. 26, 2008

Related U.S. Application Data
(60) Provisional application No. 60/976,994, filed on Oct. 2, 2007.

(51) Int. Cl. A61B 18/04; A61B 8/00

(52) U.S. Cl. 600/438; 600/439; 606/27

(57) ABSTRACT

The present invention includes an apparatus, method and system for monitoring and controlling radiation therapy, the system including a radiative source that emits energy that enters a tissue and is absorbed at or a near a target site in the tissue to heat the tissue; an ultrasound transmitter directed at the target site, wherein the ultrasound transmitter emits ultrasound signals to the tissue that has been heated by the radiative source; an ultrasound receiver directed at the target site, wherein the ultrasound receiver receives ultrasound signals emitted from the ultrasound transmitter and reflected from the tissue that has been heated by the radiative source; and a signal processor that processes the received ultrasound signal to calculate a tissue composition scan or tissue temperature scan.

### CROSS-REFERENCE TO RELATED APPLICATIONS

This application claims priority to U.S. Provisional Application Ser. No. 60/976,994, filed Oct. 2, 2007, the entire contents of which are incorporated herein by reference.

### STATEMENT OF FEDERALLY FUNDED RESEARCH

This invention was made with U.S. Government support awarded by the National Institutes of Health under grant EB 004963. The government has certain rights in this invention.

### TECHNICAL FIELD OF THE INVENTION

The present invention relates in general to the field of thermal-therapy, and more particularly, to methods and systems for the scanning, mapping, monitoring and treatment of a tissue target.

### BACKGROUND OF THE INVENTION

Without limiting the scope of the invention, its background is described in connection with tissue ablation methods and systems.

U.S. Pat. No. 6,524,250 teaches a fat layer thickness mapping system to guide liposuction surgery. This patent uses ultrasound signals to identify fat layer under the skin. Specifically impedance mis-match between layers is used to calculate different layer thickness. This technique is limited to just skin applications as it relies on knowing the order of placement of tissues, i.e. skin, followed by fat.

U.S. Pat. No. 7,060,061 teaches a method and apparatus for the selective targeting of lipid-rich tissue. This patent is about using lasers for targeting and possibly melting/destroying fat and other cosmetic purposes. Different laser wavelengths and intensities are mentioned for laser therapy. However, the laser therapy on the skin is essentially performed without pre-treatment monitoring of tissue composition and without real-time imaging of therapy progression during the treatment.

U.S. Pat. No. 7,211,044 teaches a method of mapping temperature rise using pulse-echo ultrasound. This patent shows the method of performing ultrasound based temperature mapping for ultrasound therapy. The methods described will work when the tissue composition is uniform or known a priori, e.g., in tumors or muscle. However, if the tissue composition (e.g., fat vs. water) is not known, this method will not work or provide an erroneous temperature read-out. In addition, ultrasound frames are compared before and after therapy which is not realistic as therapy is usually performed continuously and one needs to monitor temperature continuously.

U.S. Patent Application No. 20070106157 is for a non-invasive temperature estimation technique for HIFU therapy monitoring using backscattered ultrasound. This technique is used to monitor temperature during ultrasound therapy. A thermal source is used to pre-calibrate two tissue parameters (diffusivity and thermal source) which are then used to monitor temperature. The tissue under therapy is heated until the boiling point of water is reached during calibration, which could damage other tissues. In addition, since two calibration steps are needed prior to therapy, the method could be time consuming. Finally, this method is not used to differentiate between different layers like fat and muscle during or before treatment.

U.S. Patent Application No: 20070208253 teaches imaging, therapy and temperature monitoring ultrasonic system. This application uses a single ultrasound transducer for performing heating and imaging during ultrasound therapy. Temperature monitoring is performed by tracking the amplitude change in ultrasound signals. However, the amplitude of ultrasound signals has been shown to change differently for water and fatty tissues. Therefore, prior knowledge of the tissue under therapy is required to perform effective temperature monitoring which may not always be possible.

### SUMMARY OF THE INVENTION

In one embodiment, the present invention is an apparatus to monitor and control radiation therapy that includes a radiative source that emits energy that enters a tissue and is absorbed at or a near a target site in the tissue to heat the tissue; an ultrasound transmitter directed at the target site, wherein the ultrasound transmitter emits ultrasound signals to the tissue that has been heated by the radiative source; an ultrasound receiver directed at the target site, wherein the ultrasound receiver receives ultrasound signals emitted from the ultrasound transmitter and reflected from the tissue that may or may not have been heated by the radiative source; and a signal processor that processes the received ultrasound signal to calculate a tissue composition scan or tissue temperature scan. In one aspect, the invention may also include an amplifier and recorder for the reflected ultrasound signal, wherein the ultrasound signal is amplified and recorded, processed or stored to a memory device, wherein the recorder is an analog to digital converter or digitizer, and wherein the amplifier is integrated into an input of the analog to digital converter and the signal is amplified before being digitized. In another aspect, the apparatus may also include an image processor that displays a tissue composition scan to tissue temperature scan.

In operation, the radiative source can heat the tissue at, or below, a therapeutic level. In another aspect, the tissue composition scan or tissue temperature scan can be a one- (A-Scan), two- (B-Scan or M-Scan), three- (3D Scan) or four-dimensional (three space dimensions and time) dataset. In one aspect the radiative sources is selected from light, ultrasound, microwave, radio frequency or ultrasound sources. In another aspect, the ultrasound transmitter and receiver may be the same element (such as a transceiver) or two distinct elements including a transmitter and receiver. In another aspect, the transmitter and receiver may include one or more transmitters and one or more receiver elements. For example, the ultrasound transmitter may be a conventional piezoelectric transducer; a standard ultrasound array of conventional transducers, or a photoacoustic source. In another example, the ultrasound receiver may be a conventional piezoelectric transducer, a standard ultrasound array of conventional receivers or an interferometric detection system. The radiative source, the ultrasound transmitter and the ultrasound receiver may have overlapping, partially-overlapping or non-overlapping apertures.

Another embodiment of the invention is a method of generating a tissue composition scan or tissue temperature scan that includes transmitting an ultrasound signal and recording a first ultrasound scan of a tissue target; heating a targeted tissue with a radiative source; transmitting an ultrasound signal and recording a second ultrasound scan after a first radiative heating of the tissue; and generating a tissue composition scan or a tissue temperature scan, or both by calculating the time shift or amplitude change or a combination of time shift and amplitude change between the first ultrasound scan and the second ultrasound scan, wherein the ultrasound changes correlate with changes in tissue temperature variation. In one aspect, the radiative exposure is selected from a pulsed exposure (single or multi-pulse), a continuous exposure, a sub therapeutic exposure or a therapeutic exposure. The radiative source may heat the tissue at, or below, a therapeutic level. The method of the invention may also include the step of amplifying and recording the reflected ultrasound signal, wherein the ultrasound signal is amplified and recorded, processed or stored to a memory device, wherein the recorder is an analog to digital converter or digitizer, and wherein the amplifier is integrated into an input of the analog to digital converter and the signal is amplified before being digitized. Another step includes using an image processor to display a tissue composition scan or a tissue temperature scan.

In another aspect, the method of the invention may also include the steps of obtaining a tissue composition scan or a tissue temperature scan in response to a sub-therapeutic radiative exposure; and determining a therapeutic radiative dose based on the tissue composition scan or the tissue temperature scan. In another aspect, the method may include the steps of obtaining a tissue composition scan or a tissue temperature scan during a therapeutic radiative exposure; and modifying the radiative dose of the tissue target based on the tissue composition scan or the tissue temperature scan.

Yet another embodiment of the present invention includes a method and system for guiding a therapeutic regimen in real-time by transmitting and recording a first ultrasound scan of a tissue target; heating the tissue target with a radiative source; transmitting and recording a second ultrasound scan after heating the tissue; generating a tissue composition scan or a tissue temperature scan, or both by calculating the time shift or amplitude change or a combination of time shift and amplitude change between the first ultrasound scan and the second ultrasound scan, wherein the ultrasound changes correlate with changes in tissue temperature variation; determining a therapeutic radiative dose based on the tissue composition scan or the tissue temperature scan; and modifying the radiative dose of the tissue target based on the tissue composition scan or the tissue temperature scan.

### BRIEF DESCRIPTION OF THE DRAWINGS

For a more complete understanding of the features and advantages of the present invention, reference is now made to the detailed description of the invention along with the accompanying figures and in which:

FIG. 1. Apparatus setup for ultrasound, thermal and elasticity imaging during radiation therapy used to record tissue composition and temperature scans.

FIG. 2a. Temperature calibration for PVA tissue phantom; FIG. 2b. Temperature calibration for porcine muscle tissue.

FIG. 3a. Ultrasound image of tissue mimicking phantom; FIG. 3b. Tissue temperature scan after radiation therapy; and FIG. 3c. Strain image after radiation therapy.

FIG. 4a. Ultrasound image of porcine tissue; FIG. 4b. Site of laser irradiation and nanoparticle injection; FIG. 4c. Tissue temperature scan of sham therapy; and FIG. 4d. Tissue temperature scan of nanoparticle therapy.

FIG. 5. Tissue temperature scan giving increase with and without nanoparticles along the laser irradiance plane (FIG. 4a) in a sample of porcine muscle tissue.

FIG. 6a shows radiative therapy of the simulated tumor positioned at 7.5 mm depth and FIG. 6b shows the therapy of the tumor positioned at 15 mm depth.

FIG. 7. Apparatus set-up for ultrasound imaging during radiation therapy.

FIG. 8. Temperature calibration for (a) PVA tissue phantom and (b) porcine muscle tissue. The error bars represent standard deviation from 10 measurements.

FIG. 9. Radiation therapy on tissue phantoms (30 mm×30 mm). Photoabsorber embedded simulated tumor region is visible in the ultrasound image (a). Tissue temperature B-scans (b-e) recorded at 30, 60, 100 and 180 seconds respectively.

FIG. 10. Tissue temperature A-scans for tissue phantom. Spatial temperature profile (a) is along line shown in FIG. 9a. Temporal temperature profile (b) is plotted inside the therapy zone and outside the tumor as indicated by the boxes in FIG. 9a.

FIG. 11. Radiation therapy on porcine muscle tissue (20 mm×15 mm). The photoabsorber injection site with respect to the laser beam is indicated by the circle in the ultrasound image (a). Therapy performed for 120 seconds in tissue injected with water (b) at 2 W/cm² and therapy for 20 seconds in tissue injected with photoabsorbers (c-e) at 2, 3 and 4 W/cm² respectively.

FIG. 12. Tissue temperature A-scan for porcine muscle tissue. Tissue temperature A-scan (a) is along line shown in FIG. 11a for tissue injected with photoabsorbers and without photoabsorbers. Temporal A-scan illustrating the temperature profile (b) is plotted inside the therapy zone and outside it for tissue injected with nanoparticles as indicated by the boxes in FIG. 11a.

FIG. 13. Radiation therapy on porcine muscle tissue (16 mm×12 mm) at 3 W/cm2 for 3 minutes. Ultrasound B-scan before (a) and after (b) therapy indicate the therapy region. Tissue temperature B-scan (c) after 180 seconds of therapy shows therapeutic zone. Injection site is visible on the photograph (d) of the tissue sample.

FIG. 14(a) Apparatus for ultrasound imaging during radiation heating. FIG. 14(b) Digital photograph of the setup showing the orientation of the laser fiber, ex-vivo tissue and ultrasound transducer. FIG. 14(c) Block diagram for computing grayscale B-mode ultrasound image, tissue composition map and thermal image of the tissue sample.

FIG. 15(a) shows the temperature calibration for porcine fat (FIG. 15(a)) and porcine skin (FIG. 15(b)) (note, the negative gradient for fatty tissue and positive gradient for positive gradient for water-based tissue).

FIG. 16(a) shows an ultrasound image of the porcine tissue. Image covers a 10 mm (depth)×15 mm (width) region. FIG. 16(b) shows the normalized time shifts between the arrows in FIG. 16a after 1, 3 and 5 seconds of laser heating. The zero crossing indicates the dermis-fat junction.

FIG. 17(a) is a normalized time shift profile after 5 seconds of laser irradiation with clear demarcation between positive and negative normalized time shifts under laser irradiation region. FIG. 17(b) shows an ultrasound image of the porcine tissue with the zero-crossing of normalized time shifts superposed to represent the dermis-fat junction. All images represent a 10 mm×15 mm region.

FIG. 18(a) shows a thermal image showing the temperature elevation reached due to laser exposure. FIG. 18(b) shows an ultrasound image overlaid with the temperature maps, showing the temperature elevation in the dermal and fatty regions. FIG. 18(c) shows the temporal temperature rise at four regions directly along the laser irradiation plane. The regions are shown as boxes in FIG. 19b.

FIG. 19(a) shows a porcine skin overview showing defects in the subcutaneous adipose tissue (thick arrows). Wedge shaped surface lesion (thin arrows) visible showing signs of thermal denaturation of cellular structural proteins. [H & E stains. Orig. Mag. 16×]. FIG. 19(b) shows the glandular ducts show compression and are hyper chromatic. The adipose tissue (arrows) is torn and fragmented associated with subcutaneous defects. FIG. 19(c) shows normal glandular ducts surrounded by compressed fat cells (arrows) in a specimen with lower temperature increase (less than 15° C.) [H & E stains. Orig. Mag. 200×]

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Unlike the method taught in U.S. Pat. No. 6,524,250, the present invention does not use plain ultrasound signals to calculate the thickness of different layers. The signals received after a first radiative heating are processed to differentiate between different layers. Speed of sound in lipid-filled tissues (i.e. fat) and water-based tissues (e.g. muscle) changes in opposite directions in response to a temperature change. This relationship is utilized to exactly identify and measure the size of different layers. No prior knowledge of the relative order to different tissue layers or speed of sound is needed in this method. In addition, ultrasound imaging is also used to monitor tissue response to radiative surgery and temperature elevation is also computed to provide feedback for the treatment. Finally, the application is not limited to liposuction surgery, but can be applied to a variety of applications mentioned in the disclosure.

Unlike the method taught in U.S. Pat. No. 7,060,061, the present invention is about guiding and monitoring radiation therapies. Briefly, the present invention can be used to inform the user where, for how long, and with what intensity to point the radiative source (e.g., a laser) and also how not to use it. The present invention provides real-time feedback during the treatment to estimate the temperature increase in a targeted tissue region during the radiation therapy to not only damage the target tissue be it, fat, hair follicles, acne etc, but also ensure safety of skin and other non-targeted tissue structures.

Unlike the method taught in U.S. Pat. No. 7,211,044, the method of the present invention can be used to first provide a detailed composition of tissue. Next, the invention provides a tissue temperature scan continuously during therapy. In addition, ultrasound imaging can be performed during radiation therapy.

Unlike the method taught in U.S. Patent Application No. 20070106157, the method of monitoring temperature of the present invention does not utilize thermal diffusivity and a thermal source to estimate temperature change. In addition, we do not propose to perform ultrasound based-therapy; we are proposing ultrasound based monitoring during radiative (e.g., laser) therapy.

Unlike the method taught in U.S. Patent Application No: 20070208253, using the present method, the user can differentiate between different tissue types, and thus better estimate the temperature. Using the present invention, ultrasonic imaging can be performed using any combination of transmission and receiving elements. For example, it is possible to compute a tissue composition scan and a tissue temperature scan using: the speed of sound or the amplitude of ultrasound reflection. For speed of sound calculations it is possible to use the positive temperature gradient of the speed of sound for water based tissues and the negative temperature gradient of speed of sound for lipid based tissues. Alternatively, it is possible to use the amplitude of ultrasound reflections to monitor change in amplitude of ultrasound reflections caused by temperature variations. It is also possible to combine both the speed of sound and the amplitude calculations to create the tissue temperature or tissue composition scans of the present invention. Further, a laser can be used as a radiative source for generating heat, and ultrasound for monitoring the therapy.

### EXAMPLE 1

### Ultrasound-Based Thermal and Elasticity Imaging to Assist Photothermal Cancer Therapy

Photothermal therapy is a targeted, non-invasive thermal treatment of cancer. Up to 40° C. temperature increase is obtained in a small volume of malignant cells by using appropriate photoabsorbers and irradiating the tissue with a continuous wave laser. However, in order to ensure successful outcome of photothermal therapy, the tumor needs to be imaged before therapy, the temperature needs to be monitored during therapy and, finally, the tumor needs to be evaluated for necrosis during and after therapy. We investigated the feasibility of ultrasound imaging to track temperature changes during photothermal therapy and elasticity imaging to monitor tumor necrosis after treatment. The image-guided therapy was demonstrated on tissue mimicking phantoms and ex-vivo animal tissue with gold nanoparticles as photoabsorbers. Ultrasound-based thermal imaging effectively generates temperature scans during therapy while elasticity imaging monitors changes in the mechanical properties of tissue before and after therapy, allowing evaluation of treatment efficacy. Results of these study suggest that ultrasound can be used to guide photothermal therapy.

Surgery is the most direct therapeutic intervention for cancer. However, small, poorly defined lesions and tumors embedded in vital organs are difficult to treat surgically. Thermal treatments (e.g., photothermal therapy) induce a temperature increase to kill a small volume of cancerous cells and are an alternative to conventional surgery.

Photothermal therapy (PTT) is one example of the therapy that may be used with the present invention. PTT works on the principle of converting light energy into heat energy leading to tumor necrosis [1-3]. Photoabsorbers such as indocyanine green and metal nanoparticles are used in PTT to cause a selective increase in temperature. However, before PTT, the tumor must be first imaged to identify size and location of the lesion. During the therapeutic procedure, the temperature increase should be remotely monitored to ensure both tumor necrosis and protection of surrounding healthy tissue. Finally, tumor response should be inspected during and after therapy to confirm necrosis and to identify possible resurgence. Therefore, a need exists for an imaging technique that can assist, guide and monitor PTT. We propose to utilize ultrasound-based thermal and elasticity imaging to identify the tumor, to monitor temperature and tumor necrosis, and to evaluate the outcome of the photothermal therapy.

Various imaging methods including MRI, microwave radiometry, impedance tomography and ultrasound have been used for non-invasive thermal imaging. Using a real-time ultrasound imaging system, the temperature change during PTT can be estimated by measuring thermally induced differential motion of speckle. Indeed, the temperature change causes time shifts in ultrasound echo signals due to both the speed of sound change and thermal expansion of the tissue. However, if the temperature is less than 60° C., the time shifts due to the changes in the speed of sound are much larger compared to the shifts due to thermal expansion effects [4, 5]. Therefore, ultrasound imaging can be used to remotely monitor the temperature changes.

Ultrasound imaging can also be used for elasticity imaging [6]. Elasticity imaging employs the difference in tissue hardness for image contrast. The elastic properties of cancerous tissue and thermally ablated tissue can be vastly different from normal tissue. The basic principle of elasticity imaging is to use an imaging modality (e.g., ultrasound) to track the internal tissue displacement caused by an external or internal force. Multiple ultrasound frames are acquired during tissue deformation, and the induced displacements are measured by block matching or other algorithms [7]. The strain tensor is then estimated from the displacements. Finally, distribution of the tissue Young's modulus can be evaluated from the components of the displacement vector and strain tensor based on mechanical equilibrium equations [8]. Therefore, ultrasound and elasticity imaging can identify the lesion and, given PTT induced changes in mechanical properties, can monitor tumor necrosis.

Image guided photothermal therapy using ultrasound, thermal and elasticity imaging is disclosed herein. An ultrasound imaging system interfaced with continuous wave laser was assembled to perform thermal and elasticity imaging. Results from tissue/tumor mimicking phantoms and ex-vivo tissue samples demonstrate the capability of the system to monitor temperature changes and to perform elasticity imaging during photothermal therapy. In addition, a numerical model is presented to evaluate the effectiveness of PTT in cancerous tissue at different depths. The paper concludes with a discussion of image guided photothermal therapy.

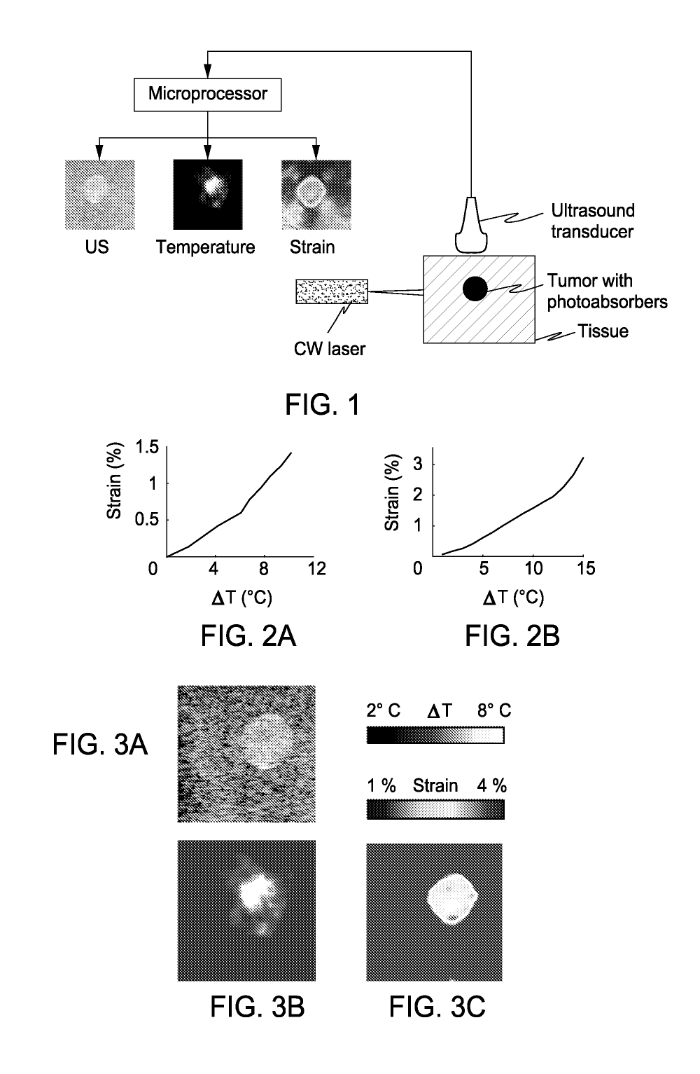

Material and methods. Apparatus set-up. The apparatus 10 setup for the image guided PTT is presented in FIG. 1. A continuous wave Nd:YAG laser 14 operating at 532 nm with a fluence of 1 J/cm² was used for photothermal therapy. A 128 element linear array transducer 12 operating at 5 mhz center frequency was used to capture ultrasound data from a tissue 16, shown here with a tumor 18. The direction of the laser beam 15 with respect to the imaging plane is shown in FIG. 1. A baseline ultrasound frame was captured before therapy. Ultrasound frames were acquired every 5 seconds during therapy, which lasted for 3 minutes. Before and after therapy, the elasticity imaging was performed by externally deforming the sample and continuously acquiring echo frames during deformation. The captured data was stored for offline processing at a microprocessor 20, which can process the data into a variety of images, e.g., ultrasound 22a, temperature 22b, and strain 22c.

For both thermal and elasticity imaging, a correlation-based block matching algorithm was employed [7]. A 0.3-mm axial and 0.9-mm lateral kernel was used to obtain an integer estimate of the displacement vector. Interpolation and phase zero-crossings were used to track sub-pixel lateral and axial displacements. Finally, axial strain was computed by using a 1-D difference filter along the axial displacement.

Sample preparation. Tissue mimicking phantoms (50×50×50 mm³) were produced using poly vinyl alcohol (PVA). PVA has modest optical absorption, scatters light similar to tissue and has speed of sound similar to tissue. Specifically, 8% PVA solution was poured into a mold and set to a desired shape by applying two freeze and thaw cycles of 12 hours each. A cylindrical, 7 mm diameter inclusion was embedded in the phantom body to mimic the tumor. Silica particles of 40-μm diameter were added in the phantom body (0.75%) and the inclusion (1.5%) for acoustic contrast. Gold nanospheres (70-nm diameter) having optical resonance around the 532 nm optical wavelength were added to the inclusion to act as photoabsorbers. Thermal imaging was performed continuously during the 3-minute long photothermal therapy while elasticity imaging was performed before and after therapy.

Tissue studies were performed using two porcine muscle tissue samples (30×30×15 mm³). The samples were immersed in water for acoustic coupling between the ultrasound transducer and tissue. Sham therapy was performed on the first sample for 3 minutes without adding nanoparticles. The second tissue sample was injected with nanoparticles (20 μl of 0.5·10¹¹ particles/ml solution) 7 mm away laterally from the site of laser irradiation, and photothermal therapy was carried out to evaluate the effect of nanoparticles on temperature rise. Thermal imaging was performed during both sham therapy and nanoparticle-enhanced therapy.

Temperature calibration. A temperature controlled water bath was used to calibrate the temperature response of the tissue mimicking phantom and samples of porcine muscle. To measure the actual temperature, a thermistor was inserted in the center of the sample. First, a baseline echo frame was captured. Then, the temperature of the water bath was increased from 24° C. to 38° C., and ultrasound frames were captures for every 1° C. temperature increment.

The time shifts at each temperature were computed in a 10 mm by 10 mm region near the thermistor using the same cross-correlation based motion tracking method [7]. Strain was then estimated from the corresponding time shifts. We assumed the temperature distribution is spatially homogenous at steady state. Thus, a strain versus temperature dependence was obtained for both the PVA phantom and porcine tissue. A nearly linear relationship was observed between temperature and induced strain (FIG. 2).

Modeling. The temperature change during PTT is due to two processes—heat generation by laser excitation and spatial redistribution by diffusion. To describe both processes, a numerical model was developed utilizing the Fourier heat equation:

$$ρ c \frac{\partial T}{\partial t} = ∇ ( λ ∇ T ) + {Q}_{s} , \qquad ( 1 )$$

where T is temperature (K), ρ is tissue density (kg/m³), c is the specific heat (J/kg/K), λ is the thermal conductivity of the tissue (W/m/K) and Qₛ (W/m³) is the external heat term.

Equation (1) was solved using explicit finite difference techniques. Monte Carlo modeling was used to calculate light propagation in a multilayered tissue model. A spherical tumor of 2 mm radius was embedded at depths of 7.5 mm and 15 mm in a homogenous medium measuring 40 mm laterally and 30 mm axially. The optical absorption coefficient (μₐ) of the tumor was varied from 30 cm⁻¹ at 7.5 mm to 900 cm⁻¹ at 15 mm while the scattering coefficient (μₛ) was 100 cm⁻¹. The homogenous tissue had an absorption coefficient of 0.8 cm⁻¹ and scattering coefficient of 10 cm⁻¹. A Gaussian beam with total power of 1 W at 808 nm was chosen to demonstrate the photothermal therapy effect at greater depths in the tissue compared to the 532 nm wavelength used in these studies.

The results of ultrasound, thermal and strain imaging in the tissue mimicking phantom are presented in FIGS. 3a-c. All images cover a 20 mm by 20 mm field of view.

The grayscale B mode image (FIG. 3a) clearly shows the tumor in an otherwise homogenous phantom. The thermal image (FIG. 3b) shows the temperature scan in the phantom immediately after photothermal therapy. The laser radiation was applied on the left side of the phantom. The calibration data (FIG. 2a) was used to convert the accumulated strain into temperature. Progressive increase in temperature during therapy was observed. The inclusion reached a temperature rise of over 7° C. at the end of therapy while the surrounding material has a temperature rise of less than 2° C. Furthermore, elasticity imaging was performed at the conclusion of PTT. The harder inclusion having lower strain is visible in the strain image (FIG. 3c). Overall, ultrasound, thermal and elasticity images show excellent co-registration.

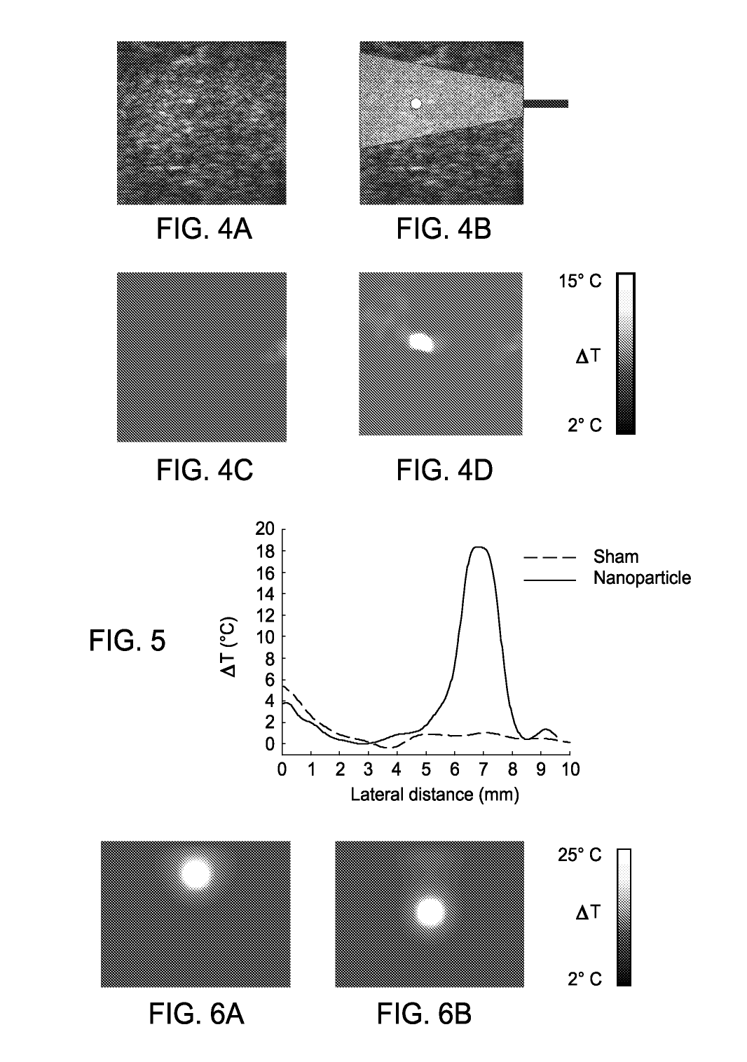

Photothermal therapy was also performed using porcine tissue. The ultrasound and thermal images (12 mm by 12 mm field of view) are presented in FIGS. 4a-d. The site of laser irradiation and nanoparticle injection is marked on the ultrasound image (FIG. 4b). Temperature increase in the sham treatment was up to 6° C. at the end of PTT (FIG. 4c). However, after nanoparticle injection, a temperature increase of over 15° C. was observed in the tissue (FIG. 4d). The approximately 2 mm diameter heated region in the nanoparticle therapy was located 7 mm inside the tissue. In sham therapy, the temperature reached maximum at the surface and gradually decreased with depth.

The injection of gold nanoparticles in tissue enhanced the photothermal therapy effects. The temperature increase in both sham and nanoparticle therapy studies along the lateral direction of laser irradiation is presented in FIG. 5. The surface temperature rise in both therapies was similar. However, a highly localized therapeutic zone with a temperature increase of over 15° C. was observed in the nanoparticle therapy at the site of photoabsorber injection.

A numerical model was constructed to evaluate the effectiveness of photothermal therapy. A laser wavelength of 808 nm was used to induce photothermal effects at varying tumor depths. A temperature increase of over 18° C. after 120 seconds of therapy was computed in tissue samples (40 mm by 30 mm field of view) with tumors positioned at 7.5 mm and 15 mm depths (FIG. 6). The surrounding tissue did not exhibit a significant temperature rise (<6° C.). To reach such temperatures in tumor and surrounding tissue with the same laser fluence and irradiation time, 1.5·10¹² particles/ml were injected in the tumor at depth 15 mm (FIG. 6b) compared to 0.5·10¹¹ particles/ml needed at a depth of 7.5 mm (FIG. 6a). Alternatively, a higher fluence or increased irradiation time could be used to target deeper tumors although this could potentially increase non-specific thermal injury of tissue in the near field.

By using appropriate photoabsorbers, a selective temperature increase was observed in the tumor with negligible temperature increase in the surrounding body. The images in FIG. 3 illustrate feasibility of using ultrasound, thermal and elasticity imaging to assist photothermal cancer therapy. By continuously monitoring the temperature distribution within the tissue, therapy progression can be tracked to prevent damage of surrounding healthy tissue. Additionally, the change in the mechanical properties of tissue due to thermal damage can be monitored. Elasticity imaging can be performed to evaluate tissue properties. In phantom studies, the temperature increase was insufficient to cause any variation in the mechanical properties of PVA. In tissue, however, the progression of the tumor necrosis can be assessed by performing elasticity imaging at regular time intervals during and after therapy.

Although laser wavelength and photoabsorber resonance were matched at 532 nm, this optical wavelength is not appropriate for tissue studies—the penetration depth in tissue is less than a few millimeters. However, there exists a near infrared (NIR) optical window of 700-1000 nm [9], where minimal light absorption in tissue leads to greater penetration depths. Various thermal coupling agents such as gold nanorods, gold nanoshells, and indocyanine green have their absorption resonance in this NIR window. Thus by using light in the NIR region with appropriate photoabsorbers, tumors at depths of a few centimeters can be treated by photothermal therapy. Our numerical studies suggest that by using a laser emitting at 808 nm with photoabsorbers resonating at the same wavelength, deep lying tumors can be treated using PTT.

Finally, a photoacoustic imaging can be used both to visualize the tumor and to monitor the therapy. Photoacoustic imaging combines the complementary properties of optics and acoustics to generate high contrast images. The same transducer can be used in ultrasound, photoacoustic and elasticity imaging [6]. The inherent differences in the optical properties of the tumor and the surrounding tissue provide the contrast for photoacoustic imaging. This contrast will be significantly enhanced by the photoabsorbers used for photothermal therapy. In addition, the pressure of photoacoustic pulse has been shown to be linearly dependent on temperature [10] and can be used to measure temperature. Thus photoacoustic imaging can be utilized to not only image the tumor but also to monitor the temperature change along with ultrasound based methods.

The results of this study strongly suggest that ultrasound can be used to image and assist photothermal therapy in real time. The results herein indicate that selective temperature increase due to photothermal therapy can be effectively monitored by ultrasound-based thermal imaging. Furthermore, elasticity imaging performed in conjunction with ultrasound adds a diagnostic tool relevant to treatment efficiency. Additionally, numerical modeling shows by using an appropriate wavelength and photoabsorbers, tumors at a reasonable depth can be treated with photothermal therapy.

### EXAMPLE 2

### Ultrasound Imaging to Monitor Photothermal Therapy Augmented by Plasmonic Nanoparticles

Metal nanoparticles are often used during photothermal therapy to efficiently convert light energy to thermal energy causing selective cancer destruction. This study investigates the feasibility of ultrasound imaging to monitor temperature changes during photothermal treatment. A continuous wave laser was used to perform photothermal therapy on tissue mimicking phantoms with embedded gold nanoparticles acting as photoabsorbers. Photothermal therapy studies were also carried out on ex-vivo tissue specimen with gold nanoparticles injected at a specific site. Prior to therapy, the structural features of the phantoms and tissue were assessed by ultrasound imaging. Thermal mapping, performed by measuring thermally induced motion of ultrasound signals, showed that temperature elevation obtained during therapy was localized to the region of embedded or injected nanoparticles. The results of our study suggest that ultrasound is a candidate approach to remotely guide nanoparticle enabled photothermal therapy.

The ability of metal nanoparticles to absorb light has greatly enhanced photothermal therapy—a technique for targeted, non-invasive cancer treatment [1-3]. Photothermal therapy relies on the principle of converting radiant energy into heat leading to tumor necrosis. These thermal treatments are an alternative to surgery for small, poorly defined lesions and tumors embedded within vital organs [4]. Simple photothermal therapy performed without exogenous photoabsorbers does not discriminate between cancer cells and surrounding tissue. In addition, high laser fluence is needed to sufficiently heat large or deeply embedded tumors. However, by using near infrared light coupled with photoabsorbers implanted in the tumor, efficient localized heating can be achieved [1-3, 5]. Temperature increases up to 40° C. were produced in nanoparticle enhanced photothermal therapy studies causing irreversible tumor damage [1-3]. A variety of metal nanoparticles including gold nanocolloids, rods or shells can be used as photoabsorbers. By varying the shape and aspect ratio, the nanoparticles can be manufactured to absorb light at the near infrared spectrum [3, 6, 7]. Photoabsorbers smaller than 200 nm have been shown to accumulate in a tumor due to a passive mechanism known as enhanced permeability and retention effect [8, 9]. Furthermore, the photoabsorbers can be bioconjugated with anti-bodies to make them tumor specific [10, 11].

For good spatial specificity, however, the tumor must first be imaged to identify the size and location of the lesion. In addition, for effective laser dosimetry the temperature increase must be remotely monitored both spatially and temporally during the procedure to ensure tumor necrosis and to protect the surrounding healthy tissue. Finally, the tumor response to therapy must be examined to confirm cancer destruction and to identify possible resurgence. Thus, a need exists for an imaging technique to plan, guide and monitor photothermal therapy. We present a preliminary investigation to utilize ultrasound imaging to identify the therapy site and monitor temperature increase.

Ultrasound has been extensively used to image and identify cancerous tissue. Recently ultrasound has been investigated to guide thermal cancer therapies including high intensity focused ultrasound [12] and radiofrequency ablation [13] by monitoring temperature. Apart from ultrasound, thermal imaging during therapy can be performed by various methods including MRI [1], microwave radiometry [14] and impedance tomography [15, 16]. However, ultrasound has several advantages—being relatively inexpensive, non-invasive and providing instantaneous feedback. Indeed, using a real time ultrasound imaging system, the temperature change during photothermal therapy can be estimated by measuring the thermally induced differential motion of speckle. When a tissue region undergoes a temperature change, the ultrasound signal experiences time shifts due to both speed of sound changing with temperature and thermal expansion of tissue [12]. To measure temperature during the therapeutic procedure, multiple ultrasound frames are acquired. The time shifts between successive ultrasound signals are then calculated by using block-matching or similar algorithms [17]. The normalized time shifts obtained and axial strains are equivalent [18]. The temperature change is directly proportional to strain and hence can be estimated by monitoring the strain image [18, 19]. Thus, ultrasound imaging can be used to monitor the temperature change.

This example demonstrates the feasibility of guiding nanoparticle enhanced photothermal therapy using ultrasound imaging. A laboratory prototype consisting of an ultrasound imaging system interfaced with a continuous wave laser was assembled to perform ultrasound-based thermal imaging during photothermal therapy. Gold nanocolloids were utilized as photoabsorbers to heat a localized region. Results from tissue/tumor mimicking phantoms and ex-vivo tissue samples demonstrate the ability of ultrasound to identify the tissue abnormalities and monitor the temperature change during therapy. A discussion of image guided photothermal therapy is provided.

Materials and Methods. Sample preparation. Photothermal therapy was first performed using 50 mm by 50 mm by 50 mm tissue/tumor phantoms constructed from poly vinyl alcohol (PVA). PVA has modest optical absorption, scatters light similarly to tissue and has been used in constructing tissue phantoms for optical imaging studies [20]. Furthermore, PVA also has speed of sound (1560 m/s at 22° C. [20]) similar to tissue. To fabricate the phantoms 8% PVA (Sigma-Aldrich, USA) solution was poured into a mold and set to a desired shape by applying two freeze and thaw cycles of 12 hours each [21]. A cylindrical 7-mm diameter inclusion was implanted within the phantom body to mimic the tumor. Silica particles (Sigma-Aldrich, USA) of 40-μm diameter were added to the phantom body (0.75% by weight) and the inclusion (1.5% by weight) for acoustic contrast. Gold nanocolloids containing 70 nm diameter nanoparticles were used the as photoabsorbers. The nanoparticles were synthesized by reducing chloroauric acid with sodium citrate [22]. The extinction maximum of the nanocolloids measured by US-Vis spectroscopy was close to 532 nm. The photoabsorbers were embedded in the inclusion. Photothermal therapy was performed by applying laser irradiance of 1 W/cm², measured at the surface of the specimen, for 3 minutes.

Ex-vivo photothermal therapy studies were performed using fresh porcine longissimus muscle. The samples, sized 30 mm by 30 mm by 15 mm, were immersed in water for acoustic coupling between the ultrasound transducer and tissue. The 20 μl solution of gold nanocolloids (0.5·10¹¹ particles/ml) was injected under ultrasound guidance using a 23-gauge hypodermic needle at an 8 mm depth from the tissue surface. The needle was inserted such that it was orthogonal to both ultrasound imaging and laser beam. The injection lasted about 12 seconds while the needle was manually held in the same position. Photothermal therapy began immediately after the injection to ensure that photoabsorbers did not diffuse through the tissue and, therefore, were localized to the injection region. Temperature rise in response to laser power density of 2, 3 and 4 W/cm² was measured by ultrasound imaging. Additionally, a control sample was injected with 20 μl of water and photothermal therapy was carried out at 2 W/cm² for 120 seconds to evaluate non-specific temperature increase.

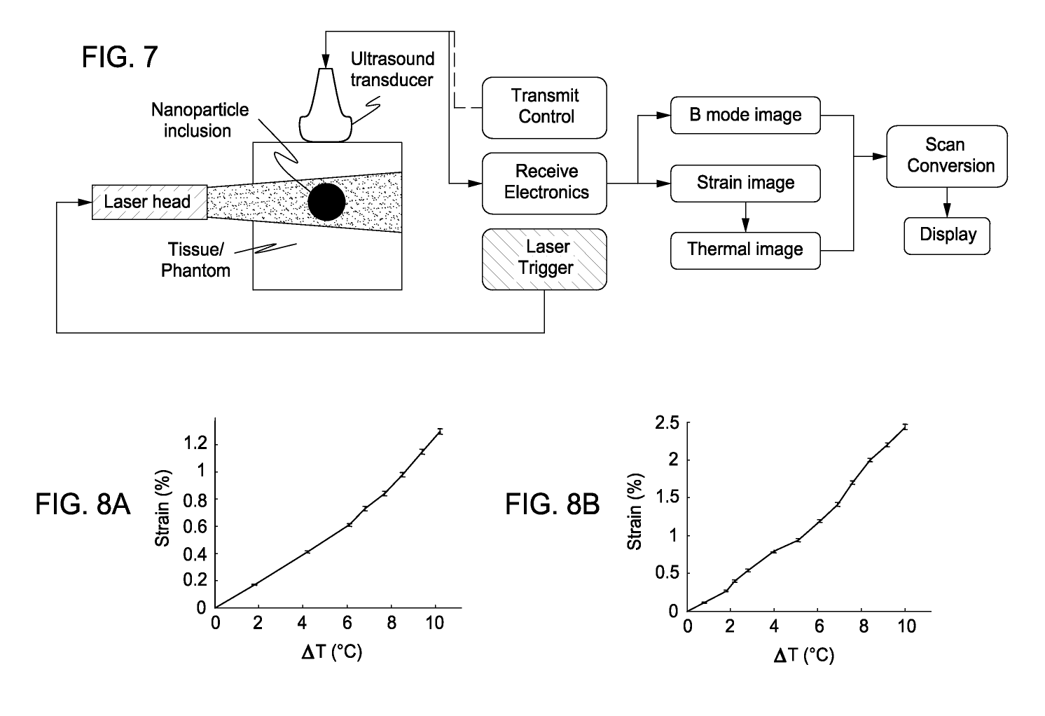

Apparatus Setup. The apparatus 10 setup for ultrasound enabled photothermal therapy (FIG. 7) utilized a frequency-doubled continuous wave Nd:YAG laser 14 with power of up to 4 W with 1 cm beam 15 diameter under control of laser trigger 28. The operating wavelength of the laser, 532-nm, was matched with the photoabsorber's optical resonance. Ultrasound imaging was performed using Sonix RP imaging system (Ultrasonix Medical Corporation, Burnaby, Canada) equipped with 128 element linear array transducer 12 operating at a 5 MHz center frequency. The RF signals were captured at 40 MHz sampling frequency (transmit control 24, receive electronics 26). Photothermal therapy studies were performed at a room temperature of 24° C. A baseline ultrasound frame was captured before therapy as a reference. During the therapeutic procedure, ultrasound frames were acquired every 5 seconds. The data was split into a B mode image 30, strain image 32 and thermal image 34. There images were converted for each scan 36 and displayed 38.

To compute a thermal image, a correlation-based block matching algorithm was performed on successive ultrasound frames offline [17]. A 0.8-mm axial and 2.1-mm lateral kernel was used—this kernel size was selected given on the trade-off between SNR and spatial resolution. A larger kernel size leads to higher signal to noise ratio (SNR) while a smaller kernel is needed for better spatial resolution [23]. Interpolation and phase zero-crossings were used to find sub-pixel lateral and axial displacements. An axial strain scan was computed using a 1.6-mm long one-dimensional difference filter along the axial displacement. Finally, the strain scan was converted to a temperature field by utilizing the strain-temperature relationship obtained from calibration studies for the PVA phantom and porcine muscle tissue.

Temperature calibration. The temperature response of both the tissue mimicking phantom and porcine muscle tissue was calibrated using a temperature controlled water bath study. A thermistor was inserted in the center of the sample to measure temperature. Initially, a baseline ultrasound frame was captured. The temperature of the water bath was then gradually increased from 24° C. to 35° C. and ultrasound frames were captured for every 1° C. temperature increment.

Temperature distribution in the sample was assumed to be spatially homogenous at steady state. Time shifts in the ultrasound signal due to temperature increase were computed in a 10 mm by 10 mm homogenous region near the thermistor. Axial strain was then estimated from the corresponding time shifts. Thus, a strain versus temperature dependence (FIGS. 8a and 8b) was obtained for the PVA phantom and porcine muscle tissue. A polynomial fit obtained for the generated curves was used to compute thermal scans from measured strain during therapy.

The apparent time shifts in ultrasound signal is primarily caused by thermally induced speed of sound change while the effect of linear expansion can be neglected for temperatures below 60° C. [24, 25]. The speed of sound linearly increases for water and water based tissues between 10-55° C. [26, 27]. Therefore, the calibration curves obtained at 24-35° C. are valid not only at physiological temperatures of 37° C. but also at temperature elevations of up to 35° C. from room temperature.

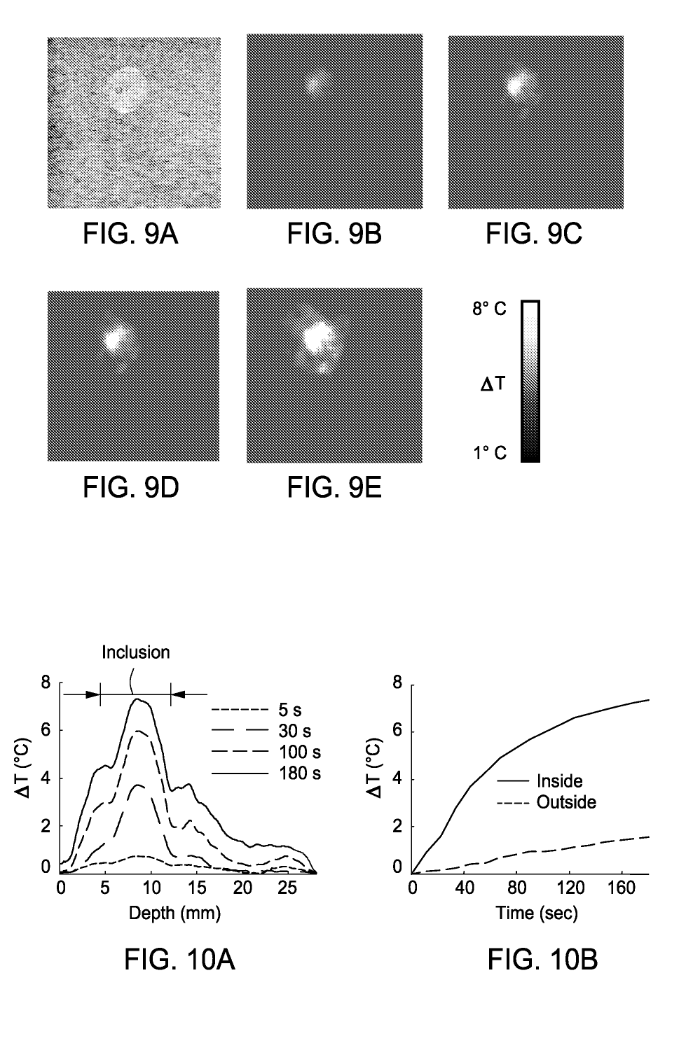

Results: Tissue/tumor phantoms. The ultrasound and computed thermal images of tissue mimicking phantom are presented in FIGS. 9a-e. All images correspond to a 30 mm by 30 mm field of view. The cylindrical inclusion modeling the tumor is easily identified in the grayscale B mode ultrasound image (FIG. 9a). Calibration data (FIG. 8a) was used to convert the accumulated strain from the captured ultrasound frames to temperature. The thermal scans (FIGS. 9b-e) after 30, 60, 100 and 180 seconds show the progressive increase in temperature. At 180 seconds, the inclusion reached a temperature increase of over 7° C. while the surrounding material has a temperature rise of less than 2° C. In these images, temperature increase is confined to the inclusion due to the presence of embedded photoabsorbers. Overall, ultrasound and the thermal images show excellent spatial co-registration.

Further examination of the thermal profile over depth and time (FIGS. 10a and 10b) shows the progressive increase in temperature. Temperature spatial profile (FIG. 10a) shows temperature rise is localized to the photoabsorber embedded inclusion. After 180 seconds of therapy, the temperature increases from a baseline of room temperature to over 7° C. above room temperature. Time-dependent temperature rise was examined in a 1.5 mm by 1.5 mm region inside and outside the inclusion (FIG. 10b). Mean temperature in the inclusion increases monotonically with time. Rate of temperature rise in the inclusion is not linear due to heat diffusion into surrounding tissue. Temperature in the region surrounding the inclusion increases with time (FIG. 10b) due to heat diffusion from the inclusion.

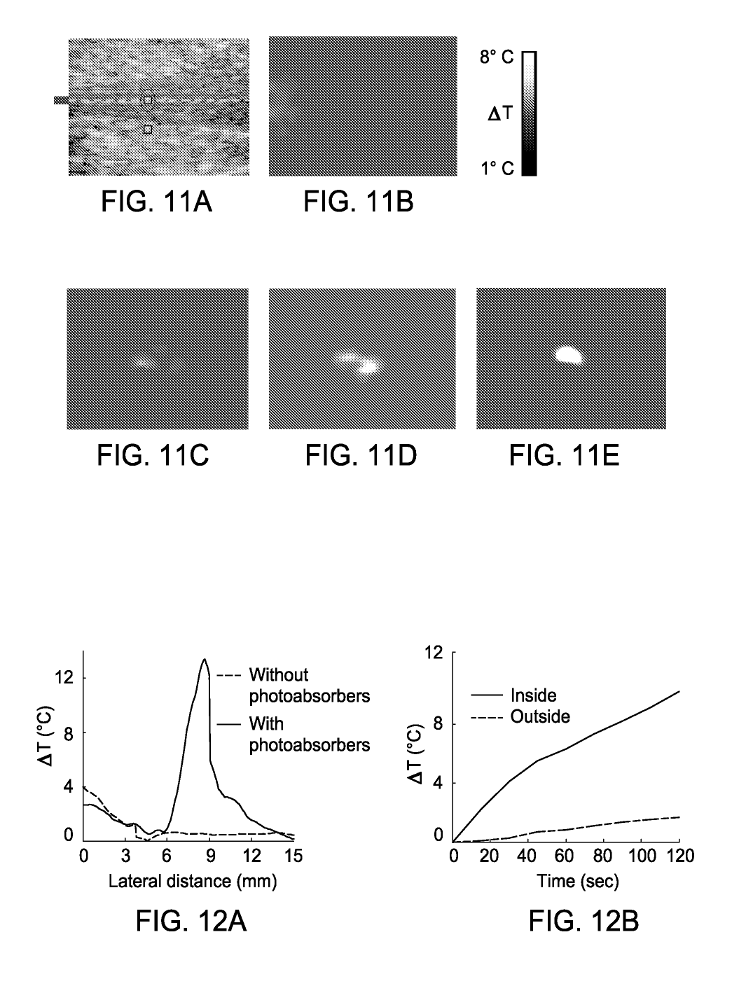

Ex-vivo tissue. Photothermal therapy was also performed on fresh porcine muscle tissue. The ultrasound and thermal images (20 mm by 15 mm field of view) are presented in FIGS. 11a-11e. The site of laser irradiation and nanoparticle injection are indicated on the ultrasound image (FIG. 11a). After 20 seconds of photothermal therapy, the temperature increased by 3° C., 6° C. and 8° C. for laser irradiances of 2, 3 and 4 W/cm² respectively (FIGS. 11c-11e). As expected, higher energies lead to a greater temperature increase. The heated region was located 8 mm from the surface of the tissue where the photoabsorbers were injected. Negligible temperature increase was observed in the control tissue where water was injected after 120 seconds of therapy (FIG. 11b).

Spatial temperature profiles were computed along the direction of laser irradiation (FIG. 12a.). Therapy was performed for 120 seconds at 2 W/cm² on two tissue samples, one containing injected nanoparticles and a control with water injected. The temperature rises by about 4° C. near the surface and then rapidly falls to below 1° C. after a depth of 3 mm for both cases. However in the tissue injected with nanoparticles, the temperature rise exceeds 12° C. at a depth of 8 mm. This therapeutic zone is highly localized and specific to the photoabsorber injection site. In the water injected tissue, no temperature rise was observed beyond 3 mm from the tissue surface. The temporal temperature profile (FIG. 12b), measured over a 1.5 mm by 1.5 mm region inside and outside the therapeutic zone was similar to that observed in the phantom study. A steady increase in average temperature with time was observed in the therapeutic zone, reaching over 10° C. after 120 seconds of therapy while the temperature outside the heated zone showed a gradual increase (1.5° C.) due to thermal diffusion.

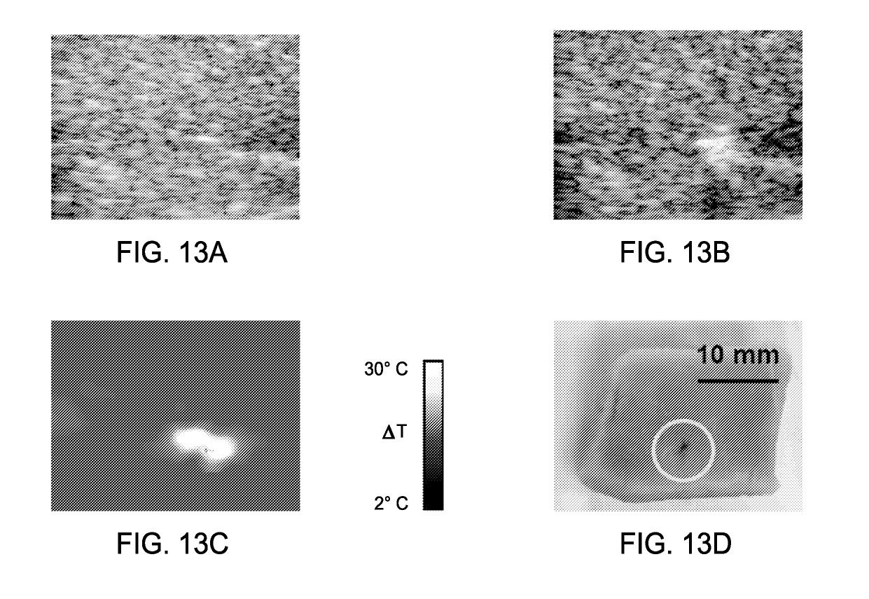

Ultrasound images recorded before and after therapy (FIGS. 13a-b, 16 mm by 12 mm field of view) demonstrate spatial location and extent of the thermal lesion created during therapy. Therapy was performed on a tissue sample at 3 W/cm² for 3 minutes. The thermal scan (FIG. 13c) shows temperature increased by over 25° C. in the therapeutic zone. There was an increase in echogenicity at the injection site after therapy accompanied by a shadow below the region of high echogenicity (FIG. 13b). By correlating the thermal image with the ultrasound image after therapy, size and location of the rounded lesion is estimated at about 3 mm in diameter and 8 mm depth. Inspection of the sample (FIG. 13d) reveals that the location of the injection site is consistent with the lesion observed in both ultrasound and thermal images.

The 532 nm optical wavelength used in this study matches the absorption spectra of the photoabsorbers. However, to carry out photothermal therapy at reasonable depths, laser irradiance in the near infrared (NIR) spectrum must be used [28]. Additionally various photoabsorbers such as gold nanorods, nanoshells, and nanocresents have their optical absorption resonance in this NIR window [3, 6, 7]. By selecting a wavelength in the NIR and appropriately matched photoabsorbers, tumors at depths of a few centimeters can be treated photothermally. Ultrasound monitoring of temperature depends on changes in the speed of sound of tissues at elevated temperatures. Using different nanoparticles does not affect the acoustic properties of tissue. Therefore, ultrasound imaging can be utilized to monitor temperature using NIR wavelengths and nanoparticles.

The temperature distribution during photothermal therapy is affected by two processes—heat generation due to absorption of laser energy and spatial redistribution of heat due to thermal diffusion. Mean temperature in the tumor with embedded nanoparticles increases with laser heating over time (FIGS. 10b and 12b). Heat diffusion results in a gradual increase in temperature around the therapeutic zone. Therefore, it is essential to monitor temperature not only within the tumor, but also in the surrounding healthy tissues. Ultrasound and thermal images (FIGS. 9 and 13) illustrate the feasibility of space-time tracking of temperature increase throughout the region of interest. Thermal scans show a progressive temperature increase in tissue undergoing therapy. The therapeutic zone was shown to be highly localized to the photoabsorber embedded region. The results indicate ultrasound-based imaging is a candidate approach to guide and monitor photothermal cancer therapy.

In the photothermal therapy studies performed here, the laser irradiation was delivered from the left side of the specimen (FIG. 7). In clinical settings, a more practical configuration is preferable with light delivery and acoustic transducer on the same side. For example, optical fibers placed along the sides of the transducer can be used for delivering the radiant energy to the tissue [29].

A pre-therapy calibration was performed to establish the relationship between apparent time shift and temperature. However, it is possible to measure temperature using a generalized and known a priori tissue specific calibration [13]. A database can be obtained to allow calculation of temperature from ultrasound time shifts directly without a calibration procedure. For tissue temperatures of 55° C. and higher, the backscattered ultrasound signal will be significantly different due to tissue state change. Under such circumstances, the temperature estimation may fail to provide accurate results. However, breakdown of ultrasound temperature monitoring may also suggest thermal damage and possibly confirm the success of treatment. Physiological motion (e.g., cardiac, respiratory) could lead to artifacts in ultrasound based temperature measurements. For example, periodic heart beats cause tissue motion which appears as time shifts on the ultrasound signal and could lead to an error in the temperature measurement. Utilizing an electrocardiogram (ECG) to trigger data capture, ultrasound frames can be collected at the same point in the cardiac cycle and thus potentially minimizing motion artifacts [30]. Along with physiological motion, operator motion of the hand-held transducer could lead to errors in temperature measurements. However, the ultrasound transducer can be secured to reduce motion artifacts as it is done in elasticity imaging, for example, where the transducer is placed in a holder [19, 31].

Finally, ultrasound can be combined with photoacoustic and elasticity imaging to form a synergistic imaging system [32, 33]. The same transducer can be used in ultrasound, photoacoustic and elasticity imaging [32, 33]. The imaging contrast in photoacoustic imaging is provided by the inherent difference in the optical properties of the tumor and surrounding tissue [34]. Photoabsorbers used during photothermal therapy significantly enhance this optical contrast [35]. Therefore, photoacoustic imaging can be used to visualize the tumor and identify the presence of photoabsorbers. Elasticity imaging on the other hand employs the difference in tissue stiffness for image contrast. Elastic properties of thermally damaged and cancerous tissue are vastly different from normal tissue [31]. Progression of tumor necrosis can be assessed using elasticity imaging at regular intervals during and after therapy [19, 31]. Ultrasound, photoacoustic and elasticity imaging can be utilized to evaluate anatomical, functional and mechanical properties of tissue during therapy, thus providing additional diagnostic tools to the clinician.

Results of this study demonstrate that ultrasound can be used to non-invasively image and guide gold nanoparticle enhanced photothermal cancer therapy. These studies show that temperature elevations of more than 20° C. can be obtained using gold nanocolloids and matching continuous wave laser. Furthermore, the temperature increase during the procedure can be monitored by ultrasound based-thermal imaging. Therapy site estimated from ultrasound and thermal images was found to be consistent with observations of gross pathology.

### EXAMPLE 3

### Ultrasound Guidance and Monitoring of Laser-Based Fat Removal

The present example used ultrasound imaging to guide laser removal of subcutaneous fat. Ultrasound imaging was used to identify the tissue composition and to monitor the temperature increase in response to laser irradiation. Laser heating was performed on ex-vivo porcine subcutaneous fat through the overlying skin using a continuous wave laser operating at 1210 nm optical wavelength. Ultrasound images were recorded using a 10 MHz linear array-based ultrasound imaging system. Ultrasound imaging was utilized to differentiate between water-based and lipid-based regions within the porcine tissue and to identify the dermis-fat junction. Temperature maps during the laser exposure in the skin and fatty tissue layers were computed. This example demonstrates the use of ultrasound imaging to guide laser fat removal.

Liposuction, also known as lipoplasty, is an invasive procedure for subcutaneous fat removal and body reshaping usually performed under local anesthesia [1]. Recent innovations in liposuction, including ultrasound and laser-assisted liposuction where fat is emulsified before applying suction [1-3], have lead to shorter treatment times and reduced scarring. Despite these advances, several disadvantages associated with liposuction are recognized such as scarring, skin sagging and risk of mortality [1,4]. Laser-based treatment for body sculpting or fat removal is a recently proposed non-invasive alternative to liposuction [5].

Selective laser heating can be achieved by utilizing an optical wavelength where the absorption by the target tissue is greater than the surrounding region [6]. Specifically for fat treatments, the absorption of lipids at vibration bands near 915, 1210 and 1720 nm exceeds that of water [5]. Using 1210 nm optical wavelength, temperature increases of greater than 20° C. were obtained and fat damage has been demonstrated through the overlying skin [5].

Prior to initiating laser therapy, knowledge of the laser dosimetry required to heat and remove the adipose tissue is required. However, the dermal-fat boundary can vary in depth from 0.5 to 4 mm while subcutaneous fat can have a thickness of a few centimeters [7]. Knowledge of the tissue composition and depth of the dermis-fat interface is useful information in selecting laser dosimetry (incident fluence, irradiation wavelength, pulse duration and exposure time).

The rupture of adipocytes has been observed in response to laser irradiation [2,3]. The mechanism leading to fat breakdown is dependent on both the heating time and the temperature increase [8,9]. During laser heating, non-specific thermal damage to the surrounding tissue is possible and may lead to scarring. For efficacious laser treatment, protecting surrounding tissue structures is essential while ensuring damage to target tissues. A need is recognized for a diagnostic imaging technique to identify the tissue composition before laser therapy and monitor the depth-resolved temperature increase during therapy.

Ultrasound is a real-time, non-invasive imaging modality that is typically employed in the diagnosis of tissue abnormalities and identification of pathological tissue [10,11]. Ultrasound imaging has also been utilized for tissue characterization based on temperature dependent changes of the speed of sound in tissue [12,13]. In addition, ultrasound imaging has been recently proposed to monitor the temperature increase in response to laser irradiation [14-16].

The setup used an ultrasound imaging system interfaced with a continuous wave laser. Studies were performed on ex-vivo porcine subcutaneous fat through the overlying epidermis and dermis. Using ultrasound imaging it was possible to both identify the dermis-fat junction and to monitor the temperature increase during therapy.

Ultrasound imaging has been used to monitor temperature changes by measuring the thermally induced change in the speed of sound [14-18]. Herein, we present a similar approach adopted to identify the tissue composition along with measurement of the temperature increase in response to laser irradiation.

The time-of-flight for ultrasound pulse-echo in a homogenous medium is given by:

$$t ( {T}_{0} ) = \frac{2 \cdot z}{c ( {T}_{0} )} , \qquad ( 1 )$$

where t(T₀) is the time delay between the transmitted pulse and an echo from a scatterer at depth z at initial temperature of T₀, and c(T₀) is the speed of sound in the medium. When the temperature rises by ΔT, an apparent time shift in arrival of the ultrasound signal is observed due to the combined effects of thermal expansion and speed of sound change. The time-of-flight for the ultrasound signal in a heated volume can be written as

$$t ( {T}_{0} + Δ T ) = \frac{2 \cdot z ( 1 + α \cdot Δ T )}{c ( {T}_{0} + Δ T )} , \qquad ( 2 )$$

where α is the linear coefficient of thermal expansion in the specimen, and c(T₀+ΔT) is the speed of sound after the temperature increase. For temperatures below 55° C. in tissue, the effect of thermal expansion on the time shift is negligible compared to the speed of sound change [19,20]. The temperature-induced apparent time shift (Δt) of the ultrasound signal can be expressed as

$$Δ t = t ( {T}_{0} + Δ T ) - t ( {T}_{0} ) = 2 \cdot [ \frac{1}{c ( {T}_{0} + Δ T )} - \frac{1}{c ( {T}_{0} )} ] \cdot z . \qquad ( 3 )$$

In water-bearing tissue, such as muscle or skin, the speed of sound increases with a rise in temperature [21]. On the other hand in lipid-based tissues, such as fat, the speed of sound decreases with a rise in temperature [21]. For example, the speed of sound in bovine liver increases with temperature at a rate of 1.83 m/(s·° C.)-comparable to that of water at 2.6 m/(s·° C.) [22]. In contrast, speed of sound decreases in bovine fat at −7.4 m/(s·° C.). Since the temperature-dependent speed of sound varies significantly between different tissue types, ultrasound-based methods for tissue characterization are possible based on general composition of water-based and lipid-based tissues [12,13]. Specifically, by tracking the apparent time shifts (Δt) in ultrasound signal arrival (which is the result of temperature-induced change in the speed of sound), subdermal fat and water/collagen rich dermis can potentially be differentiated with high contrast.

The effective temperature change can be related to the apparent time shift by the following expression

$$Δ T ( z ) = k \cdot \frac{d ( Δ t ( z ) )}{d t} , \qquad ( 4 )$$

where k is a material dependent property that can be experimentally determined, Δt(z) is the profile of the apparent time shifts between two ultrasound signals [16-18]. The term d(Δt(z))/dt is referred to as the normalized time shift and is the spatial gradient of the apparent time shifts. By computing the normalized time shifts between successive ultrasound frames (B-scans) acquired during laser heating, the spatial distribution of the temperature elevation can be determined.

A procedure is envisioned whereby a small laser induced temperature increase is produced and ultrasound imaging is used to identify regions of water- or lipid-based tissue regions (Eqs 3-4). A tissue composition map of subdermal structures can be generated by demarcating the boundary between skin and fat. During laser exposure, ultrasound imaging can be applied to estimate the spatial distribution of temperature (Eq. 4) in tissue.

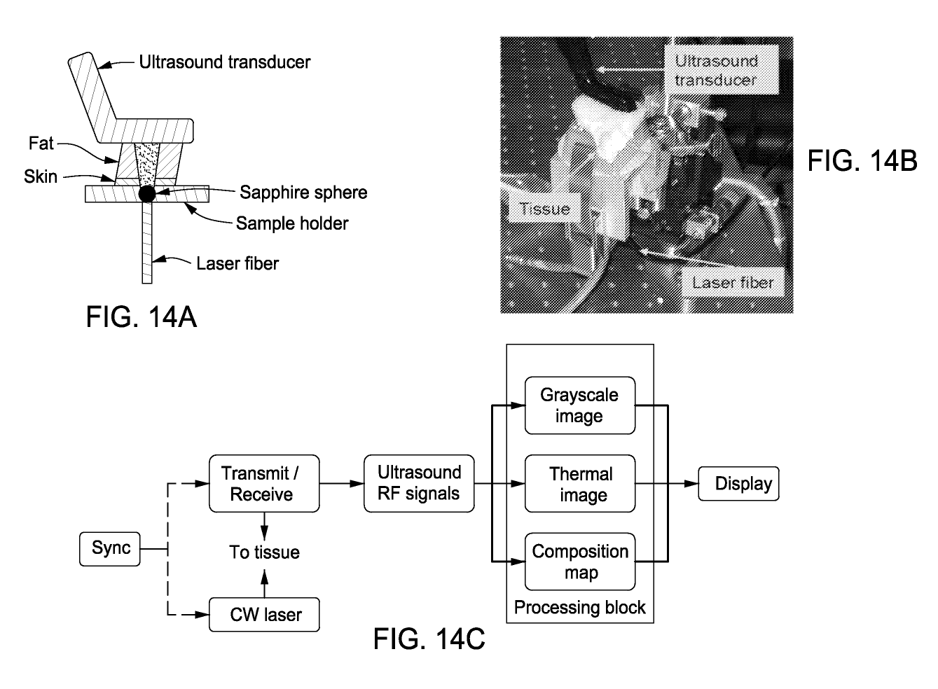

Imaging and therapeutic device. An imaging and therapeutic setup was designed and assembled to acquire ultrasound frames during laser irradiation. The diagram of the imaging and therapy setup is presented in FIG. 14a and a photograph of the assembly is shown in FIG. 14b. FIG. 14a shows a detailed view of the imaging/therapeutic device 40, shown as a cross-sectional view. A laser fiber 42 strikes sapphire sphere 44 on a sample holder 46. Skin 48 is positioned on or about the sapphire sphere 44. An ultrasound transducer 50 is positioned opposite the laser fiber 42 from the fat tissue 52. The ultrasound signals were captured using a 128 element linear transducer 50 array operating at a 10 MHz center frequency. A continuous wave laser operating at 1210 nm optical wavelength was used to deliver the radiant energy. During the laser heating, ultrasound signals were acquired every 0.1 seconds and stored for offline processing (FIG. 14c). The received signals were then used to reconstruct a grayscale B-mode image using a conventional delay-and-sum beam-forming approach. FIG. 14c shows a basic flowchart of the steps used to process the images and direct the therapeutic intervention. Briefly, the laser 60 and ultrasound transducers 62 are synchronized 64 and targeted at a tissue. The ultrasound images 66 are captured and sent to processing block 68. The processing block 68 includes three images, a gray scale image 70, thermal image 72 and a composition map 74. The user can then select which image(s) to display 78.

To obtain the tissue composition map and the thermal image, a correlation-based block matching algorithm was employed on successive ultrasound frames to estimate the apparent time shifts [23]. Then, the apparent time shifts (Eq. 3) were differentiated along the axial direction to obtain the normalized time shifts (Eq. 4). Finally, the normalized time shift profile was used to identify lipid-bearing and water-bearing tissues and to compute thermal images.

Tissue preparation. Fresh ex-vivo porcine tissue samples (15 mm×15 mm×12 mm) were obtained with skin and fat intact. The tissue samples were selected having at least 8 mm thickness of subcutaneous fat. The tissue specimen was placed on the holder (FIG. 14a) with the epidermal side on contact with a sapphire sphere of 3 mm diameter. The laser irradiation was delivered via a 300 μm diameter fiber to a sapphire sphere, which acts as a focusing lens. The ultrasound transducer was placed inline with the laser fiber gently touching the adipose-side of the tissue specimen (FIG. 14a).

The studies were performed at room temperature of 20° C. Prior to the laser irradiation, the tissue samples, which were stored in a refrigerator, were allowed to equilibrate for at least thirty minutes. The laser irradiation was applied for 5 seconds with a beam power of 0.9 W measured at the output of the fiber.

Immediately after laser irradiation, the tissue samples were bisected with a blade and fixed in formalin. Routine hematoxylin and eosin (H&E) staining was performed on the tissue slices along the laser exposure and imaging plane and observed under a light microscope.

Data analysis. Prior to laser irradiation, the temperature response of the porcine tissue was determined using a temperature controlled water bath study. Separate tissue specimens from the same animal were placed inside a constant temperature water bath. The temperature of the water bath was increased from room temperature of 20° C. to 55° C. in discrete increments. At each increment, temperature was maintained constant for thirty minutes. Then, the temperature distribution was assumed to be spatially homogenous and an ultrasound frame was recorded.

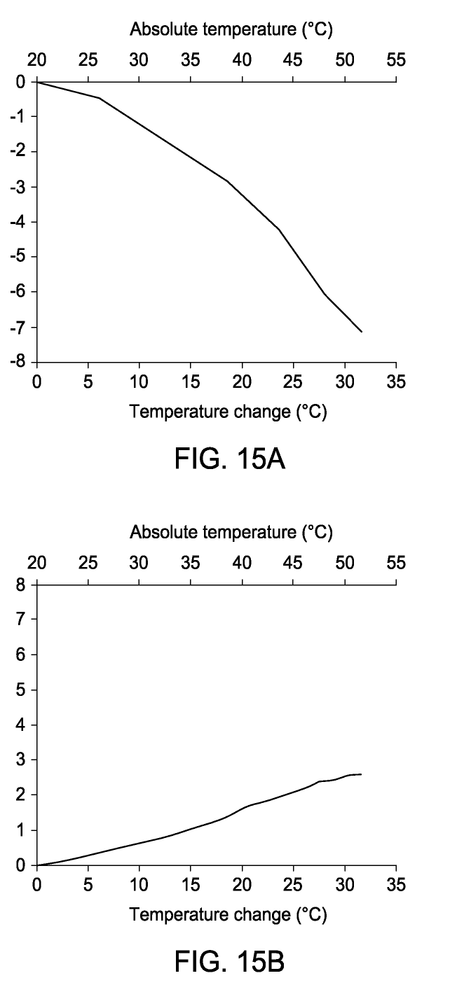

Normalized time shift profiles were computed between successive ultrasound frames from two distinct regions in the sample—fatty tissue and skin. Thus, normalized time shift vs. temperature dependence was obtained for the porcine fat and skin and was approximated using a second-order polynomial fit. The normalized time shift decreases for fatty tissue (FIG. 15a) and increases for non-fatty tissue (FIG. 15b) with temperature. Furthermore, the normalized time shift changes by a greater amount for fat (˜8%) compared to skin (˜3%) for the same temperature range. These results are consistent with literature data where the speed of sound for fat decreases while the speed of sound in water-based tissue increases with temperature [21,22].

While performing laser heating, normalized time shifts between successive ultrasound frames were estimated. The tissue composition map was then generated by identifying the sign of the gradient of the normalized time shift—negative sign indicating fat and positive sign signifying dermis. Once the tissue composition was computed and the dermal-fat junction was determined, the temperature increase was estimated by applying the coefficients of the polynomial fit (FIGS. 15a and 15b) to the measured normalized time shift.

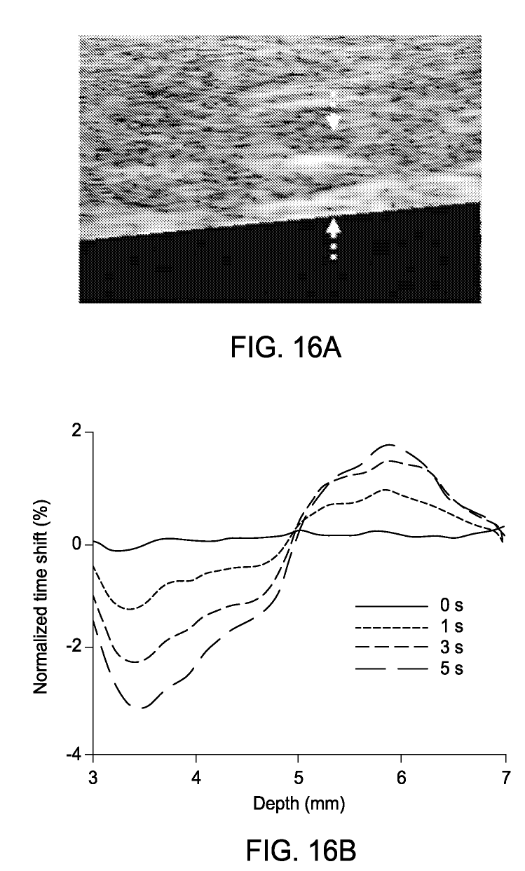

Tissue boundary map. The ultrasound image of the ex-vivo porcine tissue sample is presented in FIG. 16a representing a 10 mm×15 mm field of view. Note bottom of the ultrasound image is masked to remove reverberations of ultrasound pulse from the tissue holder.

Normalized time shift profiles were generated from successive ultrasound frames during a five second laser exposure. FIG. 16b plots the normalized time shifts along the region indicated by the arrows in FIG. 16a and represents an axial line from a depth of 3 mm to 7 mm from the top of the ultrasound image. The normalized time shifts have two distinct regions. At the 5-7 mm depth, the time shifts are increasing with the laser irradiation time while the time shifts are decreasing otherwise. Since the normalized time shift for porcine skin has positive temperature gradient while fat has a negative temperature gradient (FIG. 15), the region exhibiting a positive normalized time shift is classified as skin while the region having negative normalized time shift is classified as fat. Furthermore, the zero crossing between the positive and negative normalized time shift represents position of the dermis-fat junction. Note that location of the zero-crossing is constant regardless of the laser exposure time, i.e., the increasing magnitude of the normalized time shift does not affect position of the zero-crossing (FIG. 16b).

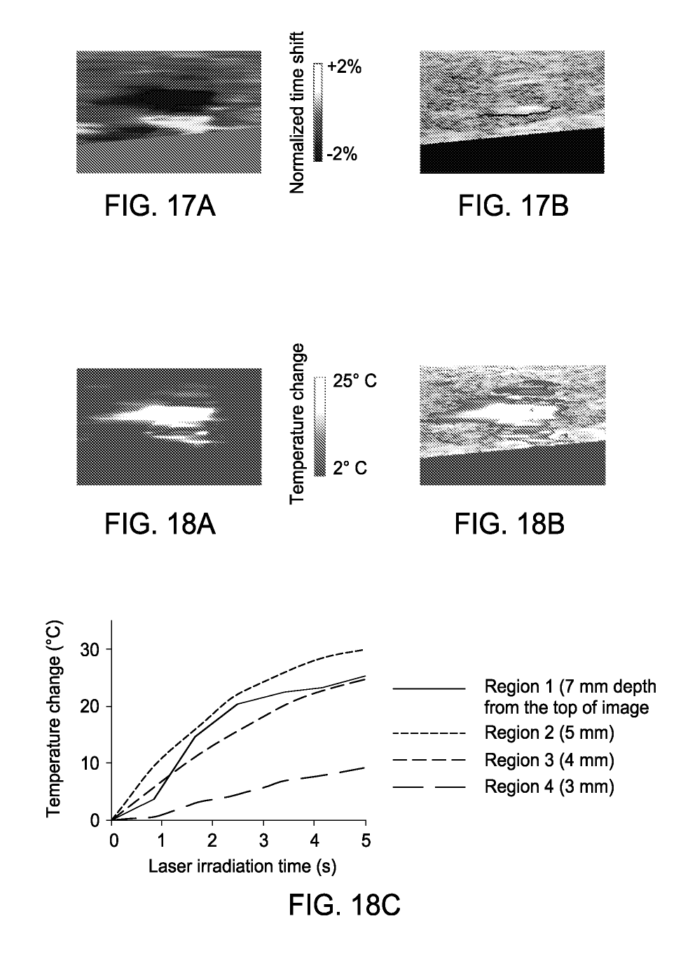

The normalized time shift profile after the 5 seconds of laser irradiation is shown in FIG. 17a. Two distinct regions are visible on the normalized time shift image above the laser irradiation spot—a brighter region having a positive normalized time shift gradient and darker region having a negative gradient. The zero-crossing between the positive and negative gradients is overlaid on the ultrasound image in FIG. 17b. The zero-crossing delineates two regions, the upper region is classified as fat and the brighter region located below is classified as skin. The zero-crossing in the normalized time shift profile may be used to identify the dermis-fat junction.

Tissue temperature map. The temperature map immediately after 5 second laser irradiation is shown in FIG. 18a. The tissue composition was first identified using the dermis-fat junction from FIG. 17b. The normalized time shifts were converted to temperature by using the respective relationships established for porcine fat (FIG. 16a) and dermis (FIG. 16b). The overlaid map (FIG. 18b) indicates that temperature increases by more than 25° C. in both skin and fatty tissue regions. In addition, the spatial-temporal temperature rise was examined in four 0.5 mm×0.5 mm regions. The regions are centered along the laser irradiation direction at depths of 7 mm, 5 mm, 4 mm and 3 mm from the top of the combined ultrasound-thermal image illustrated in FIG. 18b. Mean temperature increases monotonically with time in all four tissue regions. A temperature elevation of close to 25° C. after 5 seconds of laser irradiation is observed in region 1 located below the dermal-fat junction (i.e., in skin). No external cooling techniques were employed on the porcine tissue specimen. The temperature increases more than 30° C. in region 2—located above the dermal-fat junction and consisting primarily of fat. At deeper depths in fatty tissue (regions 3 and 4), progressively lower temperature increase is observed.

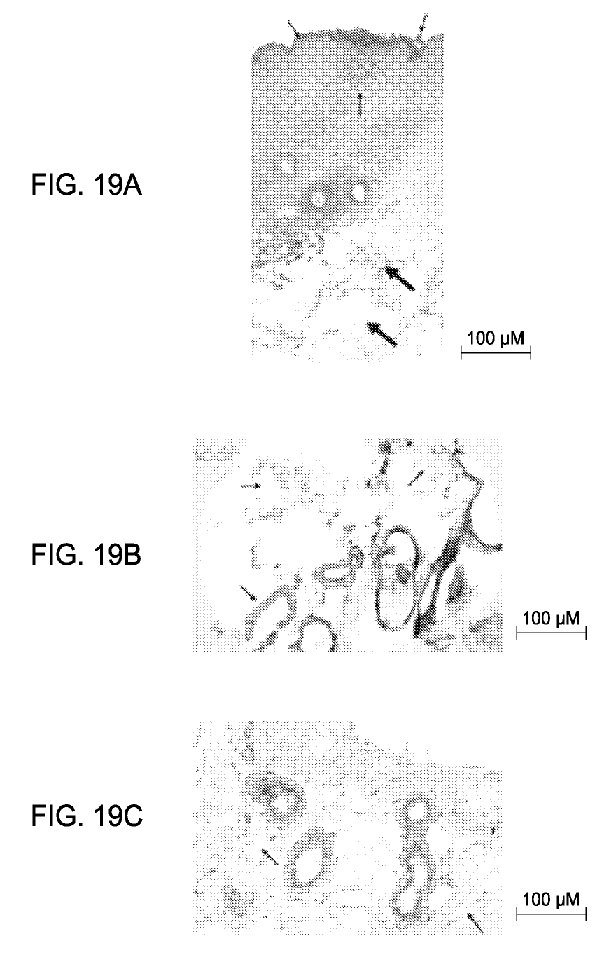

Thermal damage assessment. Histological assessment performed on the specimen shown in FIG. 18 illustrated defects in the subcutaneous adipose tissue marked with various degrees of compression, disruption and fragmentation (FIG. 19a). Distinctive thermal damage with hyalinization, swelling and loss of birefringence in the dermal collagen in a wedge-shaped region at the surface. The epithelial cells of the deeply-placed glandular ducts are shrunken and hyperchromatic while the surrounding fibroadipose tissue was torn and fragmented (FIG. 19b). In another porcine skin specimen laser heating produced a temperature rise of less than 15° C. and showed negligible thermal damage to the adipose tissue layer (FIG. 19c).

Subcutaneous fat was targeted for laser therapy by selecting a wavelength where the absorption of fat exceeds that of water [5]. However, prior to performing laser therapy for fat reduction, identifying the laser dosimetry is important. These results indicate that ultrasound imaging in combination with laser irradiation may be utilized to identify the dermis-fat junction and thereby differentiate between water-based and lipid-based tissues (FIGS. 16 and 17).

To identify the tissue composition, a small temperature increase in response to laser irradiation is needed. Since a single fiber was used to deliver the radiant energy (FIG. 15), the dermis-fat junction was identified in a relatively small (less than 7 mm) region. Using a multi-fiber delivery system or performing sequential scanning with sub-therapeutic laser dose, the entire region of interest can be safely interrogated and a complete tissue composition map generated.

To ensure irreversible thermal damage, the temperature in the therapeutic zone has to be maintained greater than 43° C. for an extended period of time [8,9]. Therefore, it is necessary to monitor the temperature increase during the laser treatment. Ultrasound-based thermal images (FIG. 18) indicate the feasibility of performing spatial and temporal mapping of temperature increase during laser irradiation.

It was found that a significant temperature increase was obtained in the dermal region (FIG. 18c). Temperature increase in the dermal region was lower than the fat region, possibly due to lipid having higher absorption coefficient as compared to water at 1210 nm laser irradiation wavelength. In a clinical application, surface cooling can be employed to protect the dermis from thermal damage [24]. Ultrasound thermal imaging may also be utilized to monitor the temperature in dermis and sub-dermal regions in response to laser irradiation.

Histological evaluation of the samples showed thermal damage at the epidermal and subjacent dermal layers in one specimen (FIGS. 19a-b). Another specimen contained subcutaneous defects that could be formed by thermal desiccation or various other mechanisms (FIG. 19c). Subcutaneous tears could be due to thermal damage and tissue desiccation possibly complicated by incomplete fixation, paraffin penetration and/or sectioning artifacts. However, distinguishing between the two mechanisms is difficult since they produce similar defects [25]. Further studies are needed to identify the laser dosimetry required to ensure thermal damage.

In this Example, laser irradiation and the ultrasound transducer were on the opposite sides of the tissue sample (FIG. 14a). This geometry led to reverberations on the bottom of the ultrasound image (FIG. 16a), introducing artifacts making tissue identification from the holder more difficult. For in vivo studies light delivery and ultrasound transducer must be on the same side. Optical fibers placed alongside the transducer can be used for delivering the radiant energy to the tissue, these setups have been assembled for photoacoustic imaging [26,27]. Another alternative is to integrate the ultrasonic transducer and the optical probe into one assembly similar to confocally arranged transducers used during high intensity focused ultrasound treatments [28].

For remote temperature assessment, a water-bath study was first performed to establish the relationship between apparent time shift and temperature (FIG. 15) for the porcine tissue sample. In ultrasound imaging temperature measurement is possible using a generalized and known a priori tissue specific calibration [18]. A database can be established to allow the calculation of temperature from ultrasound time shifts directly, accurately and in real time without a calibration procedure.

The results of this study demonstrate the ability of ultrasound imaging to guide and monitor laser therapy of fat. Ultrasound imaging was used to identify the dermis-fat boundary in porcine tissue with high contrast and to compute the temperature elevations during laser heating. Application of the ultrasound technique reported here may be relevant to clinical laser procedures to reduce fat.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES EXAMPLE 1

X. Huang, I. H. El-Sayed, W. Qian, and M. A. El-Sayed, “Cancer cell imaging and photothermal therapy in the near-infrared region by using gold nanorods,” Journal of The American Chemical Society, vol. 128, pp. 2115-20, 2006.
W. R. Chen, R. L. Adams, S. Heaton, D. T. Dickey, K. E. Bartels, and R. E. Nordquist, “Chromophore-enhanced laser-tumor tissue photothermal interaction using an 808-nm diode laser,” Cancer Letters, vol. 88, pp. 15-9, 1995.
C. Loo, A. Lin, L. Hirsch, M. H. Lee, J. Barton, N. Halas, et al., “Nanoshell-enabled photonics-based imaging and therapy of cancer,” Technology in Cancer Research & Treatment, vol. 3, pp. 33-40, 2004.
R. Maass-Moreno and C. A. Damianou, “Noninvasive temperature estimation in tissue via ultrasound echo-shifts. Part I. Analytical model,” The Journal of The Acoustical Society of America, vol. 100, pp. 2514-21, 1996.
R. Maass-Moreno, C. A. Damianou, and N. T. Sanghvi, “Noninvasive temperature estimation in tissue via ultrasound echo-shifts. Part II. In vitro study,” The Journal of The Acoustical Society of America, vol. 100, pp. 2522-30, 1996.
S. Y. Emelianov, S. R. Aglyamov, J. Shah, S. Sethuraman, W. G. Scott, R. Schmitt, et al., “Combined ultrasound, optoacoustic and elasticity imaging,” Proceedings of SPIE, vol. 5320, pp. 101-112, 2004.
M. A. Lubinski, S. Y. Emelianov, and M. O'Donnell, “Speckle tracking methods for ultrasonic elasticity imaging using short-time correlation,” IEEE Transactions on Ultrasonics, Ferroelectrics and Frequency Control, vol. 46, p. 82-96, 1999.
A. R. Skovoroda, S. Y. Emelianov, and M. O'Donnell, “Tissue elasticity reconstruction based on ultrasonic displacement and strain images,” IEEE Transactions on Ultrasonics, Ferroelectrics and Frequency Control, vol. 42, pp. 747-765, 1995.
R. Weissleder, “A clearer vision for in vivo imaging,” Nat Biotechnol, vol. 19, pp. 316-7, 2001.
I. V. Larina, K. V. Larin, and R. O. Esenaliev, “Real-time optoacoustic monitoring of temperature in tissues,” Journal of Physics D: Applied Physics, vol. 38, pp. 2633-2639, 2005.

### REFERENCES EXAMPLE 2

1. Hirsch L R, Stafford R J, Bankson J A, Sershen S R, Rivera B, Price R E, Hazle J D, Halas N J and West J L 2003 Nanoshell-mediated near-infrared thermal therapy of tumors under magnetic resonance guidance Proceedings of the National Academy of Sciences of the United States of America 100 13549-54.
2. Huang X, El-Sayed I H, Qian W and El-Sayed M A 2006 Cancer cell imaging and photothermal therapy in the near-infrared region by using gold nanorods Journal of the American Chemical Society 128 2115-20.
3. Loo C, Lowery A, Halas N, West J and Drezek R 2005 Immunotargeted nanoshells for integrated cancer imaging and therapy Nano Letters 5 709-11.
4. Goldberg S N, Gazelle G S and Mueller P R 2000 Thermal ablation therapy for focal malignancy: A unified approach to underlying principles, techniques, and diagnostic imaging guidance American Journal of Roentgenology 174 323-31.
5. Chen W R, Adams R L, Heaton S, Dickey D T, Bartels K E and Nordquist R E 1995 Chromophore-enhanced laser-tumor tissue photothermal interaction using an 808-nm diode laser Cancer Letters 88 15-9.
6. Link S and El-Sayed M A 1999 Spectral properties and relaxation dynamics of surface plasmon electronic oscillations in gold and silver nanodots and nanorods Journal of Physical Chemistry B 103 8410-26.
7. Lu Y, Liu G L, Kim J, Mejia Y X and Lee L P 2005 Nanophotonic crescent moon structures with sharp edge for ultrasensitive biomolecular detection by local electromagnetic field enhancement effect Nano Letters 5 119-24.
8. Hobbs S K, Monsky W L, Yuan F, Roberts W G, Griffith L, Torchilin V P and Jain R K 1998 Regulation of transport pathways in tumor vessels: Role of tumor type and microenvironment Proceedings of the National Academy of Sciences of the United States of America 95 4607-12.
9. Kong G, Braun R D and Dewhirst M W 2000 Hyperthermia enables tumor-specific nanoparticle delivery: Effect of particle size Cancer Research 60 4440-5.
10. Sokolov K, Follen M, Aaron J, Pavlova I, Malpica A, Lotan R and Richards-Kortum R 2003 Real-time vital optical imaging of precancer using anti-epidermal growth factor receptor antibodies conjugated to gold nanoparticles Cancer Research 63 1999-2004.
11. El-Sayed I H, Huang X and El-Sayed M A 2006 Selective laser photo-thermal therapy of epithelial carcinoma using anti-egfr antibody conjugated gold nanoparticles Cancer Letters 239 129-35.
12. Seip R and Ebbini E S 1995 Non-invasive monitoring of ultrasound phased array hyperthermia and surgery treatments Engineering in Medicine and Biology Society 1 663-4.
13. Varghese T, Zagzebski J A, Chen Q, Techavipoo U, Frank G, Johnson C, Wright A and Lee F T, Jr. 2002 Ultrasound monitoring of temperature change during radiofrequency ablation: Preliminary in-vivo results Ultrasound In Medicine & Biology 28 321-9.
14. Meaney P M, Paulsen K D and Ryan T P 1993 Microwave thermal imaging using a hybrid element method with a dual mesh scheme for reduced computation time Engineering in Medicine and Biology Society 96-7.
15. Moskowitz M J, Paulsen K D, Ryan T P and Pang D 1994 Temperature field estimation using electrical impedance profiling methods. Ii. Experimental system description and phantom results International Journal of Hyperthermia 10 229-45.
16. Paulsen K D, Moskowitz M J and Ryan T P 1994 Temperature field estimation using electrical impedance profiling methods. I. Reconstruction algorithm and simulated results International Journal of Hyperthermia 10 209-28.
17. Lubinski M A, Emelianov S Y and O'Donnell M 1999 Speckle tracking methods for ultrasonic elasticity imaging using short-time correlation IEEE Transactions on Ultrasonics, Ferroelectrics and Frequency Control 46 82-96.
18. Simon C, VanBaren P and Ebbini E S 1998 Two-dimensional temperature estimation using diagnostic ultrasound IEEE Transactions on Ultrasonics, Ferroelectrics and Frequency Control 45 1088-99.
19. Shah J, Aglyamov S R, Sokolov K, Milner T E and Emelianov S Y 2006 Ultrasound-based thermal and elasticity imaging to assist photothermal cancer therapy-preliminary study IEEE Ultrasonics Symposium 1029-32.
20. Kharine A, Manohar S, Seeton R, Kolkman R G, Bolt R A, Steenbergen W and Mul F F d 2003 Poly(vinyl alcohol) gels for use as tissue phantoms in photoacoustic mammography Physics in Medicine and Biology 48 357-70.
21. Hassan C M and Peppas N A 2000 Structure and applications of poly(vinyl alcohol) hydrogels produced by conventional crosslinking or by freezing/thawing methods Advances in polymer science 153 37-65.
22. Frens G 1973 Controlled nucleation for the regulation of the particle size in monodisperse gold suspensions Nature Physical Science 241 20-2.
23. Srinivasan S, Righetti R and Ophir J 2003 Trade-offs between the axial resolution and the signal-to-noise ratio in elastography Ultrasound in Medicine & Biology 29 847-66.
24. Maass-Moreno R and Damianou C A 1996 Noninvasive temperature estimation in tissue via ultrasound echo-shifts. Part i. Analytical model The Journal of the Acoustical Society of America 100 2514-21.
25. Maass-Moreno R, Damianou C A and Sanghvi N T 1996 Noninvasive temperature estimation in tissue via ultrasound echo-shifts. Part ii. In vitro study The Journal of the Acoustical Society of America 100 2522-30.
26. Bamber J C and Hill C R 1979 Ultrasonic attenuation and propagation speed in mammalian tissues as a function of temperature Ultrasound In Medicine and Biology 5 149-57.
27. Duck F A 1990 Physical properties of tissue Academic, New York.
28. Weissleder R 2001 A clearer vision for in vivo imaging Nature Biotechnology 19 316-7.
29. Zemp R J, Bitton R, Li M-L, Shung K K, Stoica G and Wang L V 2007 Photoacoustic imaging of the microvasculature with a high-frequency ultrasound array transducer Journal of Biomedical Optics 12 010501.
30. Simon C, VanBaren P D and Ebbini E S 1998 Motion compensation algorithm for noninvasive two-dimensional temperature estimation using diagnostic pulse-echo ultrasound SPIE-Surgical Applications of Energy 3249 182-92.
31. Varghese T, Zagzebski J A and Lee F T, Jr. 2002 Elastographic imaging of thermal lesions in the liver in vivo following radiofrequency ablation: Preliminary results Ultrasound in Medicine and Biology 28 1467-73.
32. Emelianov S Y, Aglyamov S R, Shah J, Sethuraman S, Scott W G, Schmitt R, Motamedi M, Karpiouk A and Oraevsky A A 2004 Combined ultrasound, optoacoustic and elasticity imaging SPIE Photonics West 5320 101-12.
33. Emelianov S Y, Aglyamov S R, Karpiouk A B, Mallidi S, Park S, Sethuraman S, Shah J, Smalling R W, Rubin J M and Scott W G 2006 Synergy and applications of combined ultrasound, elasticity, and photoacoustic imaging IEEE Ultrasonics Symposium 405-15.
34. Oraevsky A A, Andreev V A, Karabutov A A, Fleming D R, Gatalica Z, Singh H and Esenaliev R O 1999 Laser optoacoustic imaging of the breast: Detection of cancer angiogenesis SPIE Photonics West 3597 352-63.
35. Wang Y, Xie X, Wang X, Ku G, Gill K L, O'Neal D P, Stoica G and Wang L V 2004 Photoacoustic tomography of a nanoshell contrast agent in the in vivo rat brain Nano Letters 4 1689-92.

### REFERENCE EXAMPLE 3

1. Heymans O, Castus P, Grandjean F X, Van Zele D. Liposuction: review of the techniques, innovations and applications. Acta Chir Belg 2006; 106(6):647-653.
2. Neira R, Arroyave J, Ramirez H, Ortiz C L, Solarte E, Sequeda F, Gutierrez M I. Fat liquefaction: effect of low-level laser energy on adipose tissue. Plast Reconstr Surg 2002; 110(3):912-922; discussion 923-915.
3. Alberto G. Submental Nd:Yag laser-assisted liposuction. Lasers in Surgery and Medicine 2006; 38(3):181-184.
4. Toledo L S, Mauad R. Complications of body sculpture: prevention and treatment. Clin Plast Surg 2006; 33(1):1-11, v.
5. Anderson R R, Farinelli W, Laubach H, Manstein D, Yaroslavsky A N, III J G, Jordan K, Neil G R, Shinn M, Chandler W, Williams G P, Benson S V, Douglas D R, Dylla H F. Selective photothermolysis of lipid-rich tissues: A free electron laser study. Lasers in Surgery and Medicine 2006; 38(10):913-919.
6. Altshuler G B, Anderson R R, Manstein D, Zenzie H H, Smirnov M Z. Extended theory of selective photothermolysis. Lasers in Surgery and Medicine 2001; 29(5):416-432.
7. Illouz Y G. Study of subcutaneous fat. Aesthetic Plastic Surgery 1990; 14(1):165-177.
8. Thomsen S. Pathologic analysis of photothermal and photomechanical effects of laser-tissue interactions. Photochemistry and Photobiology 1991; 53(6):825-835.
9. Badin A Z E D, Gondek L B E, Garcia M J, Valle L Cd, Flizikowski F B Z, Noronha Ld. Analysis of laser lipolysis effects on human tissue samples obtained from liposuction Aesthetic Plastic Surgery 2005; 29(4):281-286.
10. Karlan B Y, Platt L D. Ovarian cancer screening. The role of ultrasound in early detection. Cancer 1995; 76:2011-2015.
11. Teh W, Wilson A R M. The role of ultrasound in breast cancer screening. A consensus statement by the European Group for breast cancer screening. European Journal of Cancer 1998; 34(4):449-450.
12. Pereira F R, Machado J C, Foster F S. Ultrasound characterization of coronary artery wall in vitro using temperature-dependent wave speed. Ultrasonics, Ferroelectrics and Frequency Control, IEEE Transactions on 2003; 50(11):1474-1485.
13. Shi Y, Witte R S, Milas S M, Neiss J H, Chen X C, Cain C A, O'Donnell M. Ultrasonic thermal imaging of microwave absorption. Proceeding of the 2003 IEEE Ultrasonics Symposium 2003; 1:224-227
14. Shah J, Aglyamov S R, Sokolov K, Milner T E, Emelianov S Y. Ultrasound-based thermal and elasticity imaging to assist photothermal cancer therapy—Preliminary study. Proceeding of the 2006 IEEE Ultrasonics Symposium 2006:1029-1032.
15. Shah J, Park S, Aglyamov S, Larson T, Ma L, Sokolov K, Johnston K, Milner T, Emelianov S. Photoacoustic and ultrasound imaging to guide photothermal therapy: ex-vivo study. Proc SPIE 2008; 6856:68560 U.
16. Shah J, Aglyamov S R, Sokolov K, Milner T E, Emelianov S Y. Ultrasound imaging to monitor photothermal therapy—Feasibility study. Optics Express 2008; 16(6):3776-3785.
17. Seip R, Ebbini E S. Non-invasive monitoring of ultrasound phased array hyperthermia and surgery treatments. 1995. p 663-664 vol. 661.
18. Varghese T, Zagzebski J A, Chen Q, Techavipoo U, Frank G, Johnson C, Wright A, Lee F T, Jr. Ultrasound monitoring of temperature change during radiofrequency ablation: preliminary in-vivo results. Ultrasound Med Biol 2002; 28(3):321-329.
19. Maass-Moreno R, Damianou C A. Noninvasive temperature estimation in tissue via ultrasound echo-shifts. Part I. Analytical model. The Journal of the Acoustical Society of America 1996; 100(4 Pt 1):2514-2521.
20. Maass-Moreno R, Damianou C A, Sanghvi N T. Noninvasive temperature estimation in tissue via ultrasound echo-shifts. Part II. In vitro study. The Journal of the Acoustical Society of America 1996; 100(4 Pt 1):2522-2530.
21. Duck F A. Physical properties of tissue: Academic, New York. 1990.
22. Bamber J C, Hill C R. Ultrasonic attenuation and propagation speed in mammalian tissues as a function of temperature. Ultrasound in Medicine and Biology 1979; 5:149-157.
23. Lubinski M A, Emelianov S Y, O'Donnell M. Speckle tracking methods for ultrasonic elasticity imaging using short-time correlation. Ultrasonics, Ferroelectrics and Frequency Control, IEEE Transactions on 1999; 46(1):82-96.
24. Pfefer T J, Smithies D J, Milner T E, Gemert M J Cv, Nelson J S, Welch A J. Bioheat transfer analysis of cryogen spray cooling during laser treatment of port wine stains. Lasers in Surgery and Medicine 2000; 26(2):145-157.
25. Thomsen S L. Practical pathology for engineers: how to do the job right the first time. Proceedings of SPIE 2003; 4954(1):476635.
26. Zemp R J, Bitton R, Li M-L, Shung K K, Stoica G, Wang L V. Photoacoustic imaging of the microvasculature with a high-frequency ultrasound array transducer. Journal of Biomedical Optics 2007; 12(1):010501.
27. Park S, Mallidi S, Karpiouk A B, Aglyamov S, Emelianov S Y. Photoacoustic imaging using array transducer. Proc SPIE 2007; 6437:643714.
28. Seip R, Ebbini E S. Noninvasive estimation of tissue temperature response to heating fields using diagnostic ultrasound. Biomedical Engineering, IEEE Transactions on 1995; 42(8):828-839.

What is claimed is:

1. An apparatus to monitor and control radiation therapy comprising:
a radiative source that emits energy that enters a tissue and is absorbed at or a near a target site in the tissue to heat the tissue;
an ultrasound transmitter directed at the target site, wherein the ultrasound transmitter emits ultrasound signals to the tissue that has been heated by the radiative source;
an ultrasound receiver directed at the target site, wherein the ultrasound receiver receives ultrasound signals emitted from the ultrasound transmitter and reflected from the tissue that has been heated by the radiative source; and
a signal processor that processes the received ultrasound signals to calculate a tissue composition scan or tissue temperature scan.

2. The apparatus of claim 1, further comprising an amplifier and recorder for the reflected ultrasound signal, wherein the ultrasound signal is amplified and recorded, processed or stored to a memory device, wherein the recorder is an analog to digital converter or digitizer, and wherein the amplifier is integrated into an input of the analog to digital converter and the signal is amplified before being digitized.

3. The apparatus of claim 1, further comprising an image processor that displays a tissue composition scan or a tissue temperature scan.

4. The apparatus of claim 1, wherein the radiative source heats the tissue at, or below, a therapeutic level.

5. The apparatus of claim 1, wherein tissue composition scan or tissue temperature scan comprises a one- (A-Scan), two- (B-Scan or M-Scan), three- (3D-Scan) or four- (three space dimensions and time) dimensional scan dataset of the tissue composition scan or the tissue temperature scan.

6. The apparatus of claim 1, wherein the radiative sources is selected from light, microwave, radio frequency or ultrasound sources.

7. The apparatus of claim 1, wherein the ultrasound transmitter and receiver may be the same element (such as a transceiver) or two distinct elements including a transmitter and receiver.

8. The apparatus of claim 1, wherein the ultrasound transmitter and receiver comprise one or more transmitter and one or more receiver elements.

9. The apparatus of claim 1, wherein the ultrasound transmitter comprises a conventional piezoelectric transducer; a standard ultrasound array of conventional transducers, or a photoacoustic source.

10. The apparatus of claim 1, wherein the ultrasound receiver comprises a conventional piezoelectric transducer, a standard ultrasound array of conventional receivers or an interferometric detection system.

11. The apparatus of claim 1, wherein the radiative source, the ultrasound transmitter and the ultrasound receiver have overlapping, partially-overlapping or non-overlapping apertures.

12. A method of generating a tissue composition scan or tissue temperature scan comprising:
transmitting an ultrasound signal and recording a first ultrasound scan of a tissue target; heating a targeted tissue with a radiative source;
transmitting an ultrasound signal and recording a second ultrasound scan after or during a first radiative heating of the tissue; and
generating a tissue composition scan or a tissue temperature scan, or both by calculating the difference between the first ultrasound scan and the second ultrasound scan or an accumulation of multiple successive ultrasound scans, wherein the ultrasound changes correlate with changes in tissue temperature variation.

13. The method of claim 11, wherein the radiative exposure is selected from a pulsed exposure (single or multi-pulse), a continuous exposure, a therapeutic exposure or a sub therapeutic exposure.

14. The method of claim 11, wherein the radiative source heats the tissue at, or below, a therapeutic level.

15. The method of claim 11, further comprising the step of amplifying and recording the reflected ultrasound signal, wherein the ultrasound signal is amplified and recorded, processed or stored to a memory device, wherein the recorder is an analog to digital converter or digitizer, and wherein the amplifier is integrated into an input of the analog to digital converter and the signal is amplified before being digitized.

16. The method of claim 11, further comprising the step using an image processor to display a tissue composition scan or a tissue temperature scan.

17. The method of claim 11, wherein tissue composition scan or tissue temperature scan comprises a one- (A-Scan), two- (B-Scan or M-Scan), three- (3D-Scan) or four- (three space and one time dimension) dimensional dataset.

18. The method of claim 11, wherein the radiative source or sources is selected from light, ultrasound, microwave, radio frequency or ultrasound sources.

19. The method of claim 11, wherein the ultrasound signal transmitter and receiver may be the same element or two distinct elements.

20. The method of claim 11, wherein the ultrasound transmitter and receiver comprise one or more transmitter or receiver elements.

21. The method of claim 11, wherein the ultrasound transmitter comprises an ultrasound transmitter; a conventional piezoelectric transducer; a standard ultrasound array of conventional transducers, a photoacoustic source or an interferometric source.

22. The method of claim 11, wherein the radiative source, the ultrasound transmitter and the ultrasound receiver have overlapping, partially-overlapping or non-overlapping apertures.

23. The method of claim 11, further comprising the steps of:
obtaining a tissue composition scan or a tissue temperature scan in response to a sub-therapeutic radiative exposure; and
determining a therapeutic radiative dose based on the tissue composition scan or the tissue temperature scan.

24. The method of claim 11, further comprising the steps of:
obtaining a tissue composition scan or a tissue temperature scan during a therapeutic radiative exposure; and
modifying the radiative dose of the tissue target based on the tissue composition scan or the tissue temperature scan.

25. A method of guiding a therapeutic regimen in real-time comprising:
transmitting and recording a first ultrasound scan of a tissue target;
heating the tissue target with a radiative source without thermal denaturation of tissue proteins;
transmitting and recording a second ultrasound scan after or during heating the tissue;
generating a tissue composition scan or a tissue temperature scan, or both by calculating the difference between the first ultrasound scan and the second ultrasound scan or an accumulation of multiple successive ultrasound scans, wherein the ultrasound changes correlate with changes in tissue temperature variation;
determining a therapeutic radiative dose based on the tissue composition scan or the tissue temperature scan; and
modifying the radiative dose of the tissue target based on the tissue composition scan or the tissue temperature scan.
